SEQUENCE LISTING (25 sequences)

SEQ ID NO: 1 - protein, 13 aa, Artificial Sequence
  Laboratory-synthesized sequence
AHIVMVDAYKPTK

SEQ ID NO: 2 - protein, 134 aa, Artificial Sequence
  Laboratory-synthesized sequence
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 3 - protein, 15 aa, Artificial Sequence
  Laboratory-synthesized sequence
GLNDIFEAQKIEWHE

SEQ ID NO: 4 - protein, 11 aa, Artificial Sequence
  Laboratory-synthesized sequence
DSLEFIASKLA

SEQ ID NO: 5 - protein, 22 aa, Artificial Sequence
  Laboratory-synthesized sequence
DEVLVEIETDKAVLEVPGGEEE

SEQ ID NO: 6 - protein, 13 aa, Artificial Sequence
  Laboratory-synthesized sequence
GFEIDKVWYDLDA

SEQ ID NO: 7 - DNA, 29 bp, Artificial Sequence
  Laboratory-synthesized sequence
aatgatacggcgaccaccgagatctacac

SEQ ID NO: 8 - DNA, 24 bp, Artificial Sequence
  Laboratory-synthesized sequence
caagcagaagacggcatacgagat

SEQ ID NO: 9 - DNA, 30 bp, Artificial Sequence
  Laboratory-synthesized sequence
gaatgccgcttacagtacgcctaggtcagt

SEQ ID NO: 10 - DNA, 30 bp, Artificial Sequence
  Laboratory-synthesized sequence
tccgactaagccagtaagcggcattccagt

SEQ ID NO: 11 - DNA, 65 bp, Artificial Sequence
  Laboratory-synthesized sequence
acctaggcgtacttggcttagtcggattttttttttgtgtagatctcggtggtcgccgtatcatt

SEQ ID NO: 12 - DNA, 58 bp, Artificial Sequence
  Laboratory-synthesized sequence
aatgatacggcgaccaccgagatctacactctttccctacacgacgctcttccgatct

SEQ ID NO: 13 - DNA, 30 bp, Artificial Sequence
  Laboratory-synthesized sequence
gaatgccgcttacagtacgcctaggtactg

SEQ ID NO: 14 - DNA, 30 bp, Artificial Sequence
  Laboratory-synthesized sequence
tccgactaagccagtaagcggcattccgat

SEQ ID NO: 15 - DNA, 30 bp, Artificial Sequence
  Laboratory-synthesized sequence
acctaggcgtacttggcttagtcggacgat

SEQ ID NO: 16 - DNA, 30 bp, Artificial Sequence
  Laboratory-synthesized sequence
gaatgccgcttacagtacgcctaggtatcg

SEQ ID NO: 17 - DNA, 30 bp, Artificial Sequence
  Laboratory-synthesized sequence
tccgactaagccagtaagcggcattcgcat

SEQ ID NO: 18 - DNA, 30 bp, Artificial Sequence
  Laboratory-synthesized sequence
acctaggcgtacttggcttagtcggagcat

SEQ ID NO: 19 - DNA, 30 bp, Artificial Sequence
  Laboratory-synthesized sequence
gaatgccgcttacagtacgcctaggtatgc

SEQ ID NO: 20 - DNA, 30 bp, Artificial Sequence
  Laboratory-synthesized sequence
tccgactaagccagtaagcggcattcttgc

SEQ ID NO: 21 - DNA, 30 bp, Artificial Sequence
  Laboratory-synthesized sequence
acctaggcgtacttggcttagtcggattgc

SEQ ID NO: 22 - DNA, 30 bp, Artificial Sequence
  Laboratory-synthesized sequence
gaatgccgcttacagtacgcctaggtgcaa

SEQ ID NO: 23 - DNA, 30 bp, Artificial Sequence
  Laboratory-synthesized sequence
tccgactaagccagtaagcggcattcggat

SEQ ID NO: 24 - DNA, 30 bp, Artificial Sequence
  Laboratory-synthesized sequence
acctaggcgtacttggcttagtcggaggat

SEQ ID NO: 25 - DNA, 24 bp, Artificial Sequence
  Laboratory-synthesized sequence
cctcctcctcctcctcctcctcct

(19) United States
(12) Patent Application Publication
(10) Pub. No.: US 2021/0187469 A1
(43) Pub. Date: Jun. 24, 2021

(54) NANOPARTICLE WITH SINGLE SITE FOR TEMPLATE POLYNUCLEOTIDE ATTACHMENT

(71) Applicants: ILLUMINA, INC., San Diego, CA (US); ILLUMINA CAMBRIDGE LIMITED, Cambridge (GB); ILLUMINA SINGAPORE PTE. LTD., Singapore (SG)

(72) Inventors: Wayne N. GEORGE, Cambridge (GB); Jonathan Mark BOUTELL, Cambridge (GB); Mathieu LESSARD-VIGER, San Diego, CA (US); Saurabh NIRANTAR, Singapore (SG); Pietro GATTI LAFRANCONI, Cambridge (GB); Seth MCDONALD, San Diego, CA (US); Yin Nah TEO, Singapore (SG); Maria Rogert BACIGALUPO, San Diego, CA (US); Sergio PEISAJOVICH, San Diego, CA (US); Eric BRUSTAD, San Diego, CA (US); Michael HOWARD, San Diego, CA (US); Xiangyuan YANG, Singapore (SG); M. Shane Bowen, San Diego, CA (US); Alex NEMIROSKI, San Diego, CA (US); Jeffrey BRODIN, San Diego, CA (US); Ludovic VINCENT, San Diego, CA (US); Lorenzo BERTI, San Diego, CA (US); Andrew A. BROWN, Cambridge (GB); Fei SHEN, San Diego, CA (US); Lena STORMS, San Diego, CA (US); Olivia BENICE, San Diego, CA (US)

(73) Assignees: ILLUMINA, INC., San Diego, CA (US); ILLUMINA CAMBRIDGE LIMITED, Cambridge (GB); ILLUMINA SINGAPORE PTE. LTD., Singapore (SG)

(21) Appl. No.: 17/130,489

(22) Filed: Dec. 22, 2020

Related U.S. Application Data
(60) Provisional application No. 62/952,799, filed on Dec. 23, 2019.
(60) Provisional application No. 62/952,866, filed on Dec. 23, 2019.

(51) Int. Cl. B01J 19/00

(52) U.S. Cl. CPC B01J 19/0046; B82Y 30/00

(57) ABSTRACT

Provided is a nanoparticle including a scaffold, a single template site for bonding a template polynucleotide to the scaffold, and a plurality of accessory sites for bonding accessory oligonucleotides to the scaffold, wherein the scaffold is selected from one or more scaffold DNA molecules and one or more scaffold polypeptides, the single template site for bonding a template polynucleotide to the scaffold is selected from a covalent template bonding site and a noncovalent template bonding site and the plurality of accessory sites for bonding accessory oligonucleotides to the scaffold are selected from covalent accessory oligonucleotide bonding sites and noncovalent accessory oligonucleotide bonding sites. Also provided are methods of using the nanoparticle.

### CROSS-REFERENCE TO RELATED APPLICATIONS

This application claims benefit of priority from U.S. Provisional Patent Application No. 62/952,799, filed on Dec. 23, 2019, and U.S. Provisional Patent Application No. 62/952,866, filed on Dec. 23, 2019, the entire contents of which are incorporated herein by reference.

### SEQUENCE LISTING

The instant application contains a Sequence Listing, created on Dec. 16, 2020; the file, in ASCII format, is designated H1912656.txt and is 6.8 KB in size. The file is hereby incorporated by reference in its entirety into the instant application.

### BACKGROUND

Many current sequencing platforms use “sequencing by synthesis” (SBS) technology and fluorescence-based methods for detection. In some examples, numerous target polynucleotides isolated from a library to be sequences, or template polynucleotides, are attached to a surface of a substrate in a process known as seeding. Multiple copies of the template polynucleotides may then be synthesized in attachment to the surface in proximity to where a template polynucleotide of which it is a copy was seeded, in a process called clustering. Subsequently, nascent copies of the clustered polynucleotides are synthesized under conditions in which they emit a signal identifying each nucleotide as it is attached to the nascent strand. Clustering of a plurality of copies of the seeded template polynucleotide in proximity to where it was initially seeded results in amplification of signal generated during the visualizable polymerization, improving detection.

Seeding and clustering for SBS work well when as much of an available substrate surface as possible is seeded by template polynucleotides, which may maximize an amount of sequencing information obtainable during a sequencing run. By contrast, generally speaking the less available surface area of a substrate used for seeding and clustering, the less efficient an SBS process may be, resulting in increased time, reactants, expense, and complicated data processing for obtaining a given amount of sequencing information of a given library.

Seeding and clustering also work well when template polynucleotides from a library with sequences that differ from each other seed on, or attach to, positions of the surface sufficiently distal from each other such that clustering results in spatially distinct clusters of copied polynucleotides each resulting from the seeding of a single template polynucleotide, a condition generally referred to as monoclonality. That is, a library of template polynucleotides may generally include a high number of template polynucleotide molecules whose nucleotide sequences differ from each other's. If two such template polynucleotides seed too closely together on a surface of a substrate, clustering may result in spatially comingled populations of copied polynucleotides, some of which having a sequence of one of the template polynucleotides that seeded nearby and others having a sequence of another template polynucleotide that also seeded nearby on the surface. Or, two clusters formed from two different template polynucleotides that seeded in too close proximity to each other may be too adjacent to each other or adjoin each other such that an imaging system used in an SBS process may be unable to distinguish them as separate clusters even though there may be no or minimal spatial comingling of substrate-attached sequences between the clusters. Such a disadvantageous condition may generally be referred to as polyclonality. It may be more difficult, time consuming, expensive, and less efficient, and require more complicated data analytics to obtain unambiguous sequence information from a polyclonal cluster if present.

### SUMMARY

It is therefore desirable to perform SBS under conditions under which as much available surface area as possible of a substrate surface is used for seeding and clustering, while also promoting separation of seeded template polynucleotides so as to maximize monoclonality of clusters as possible and minimize polyclonal clusters as much as possible. Disclosed herein are compositions and methods that may be used for advantageously increasing seeding density and monoclonal clustering in SBS.

In one aspect, provided is a nanoparticle, including a scaffold, a single template site for bonding a template polynucleotide to the scaffold, and a plurality of accessory sites for bonding accessory oligonucleotides to the scaffold, wherein the scaffold is selected from one or more scaffold DNA molecules and one or more scaffold polypeptides, the single template site for bonding a template polynucleotide to the scaffold is selected from a covalent template bonding site and a noncovalent template bonding site, and the plurality of accessory sites for bonding accessory oligonucleotides to the scaffold is selected from covalent accessory oligonucleotide bonding sites and noncovalent accessory oligonucleotide bonding sites.

In an example, the scaffold includes one or a plurality of scaffold DNA molecules. In another example, the scaffold includes a plurality of scaffold DNA molecules, wherein the plurality of scaffold DNA molecules comprises a DNA dendrimer. In yet another example, the DNA dendrimer includes a number of generations of bifurcating constitutional repeating units wherein the number of generations is from 2 to 100. In still another example, the bifurcating constitutional repeating units each include three constitutional repeating unit oligodeoxyribonucleotides hybridized to each other to form an adapter including one upstream overhang and two downstream overhangs, wherein the upstream overhang of each adapter in generation 2 and higher is complementary to a downstream overhang of an immediately upstream constitutional repeating unit, and the downstream overhang of the adapter in generation 1 includes the single template site. In a further example, the scaffold includes a single-stranded DNA.

In another example, the scaffold includes one or more scaffold polypeptide. In another example, the scaffold polypeptide includes a green fluorescent protein.

In another example, the single template site includes a covalent template bonding site. In yet another example, the covalent template bonding site is selected from an amine-NETS ester bonding site, an amine-imidoester bonding site, an amine-pentofluorophenyl ester bonding site, an amine-hydroxymethyl phosphine bonding site, a carboxyl-carbodiimide bonding site, a thiol-maleimide bonding site, a thiol-haloacetyl bonding site, a thiol-pyridyl disulfide bonding site, a thiol-thiosulfonate bonding site, a thiol-vinyl sulfone bonding site, an aldehyde-hydrazide bonding site, an aldehyde-alkoxyamine bonding site, a hydroxy-isocyanate bonding site, an azide-alkyne bonding site, an azide-phosphine bonding site, a transcyclooctene-tetrazine bonding site, a norbornene-tetrazine bonding site, an azide-cyclooctyne bonding site, an azide-norbornene bonding site, an oxime bonding site, a SpyTag-SpyCatcher bonding site, a Snap-tag-O⁶-Benzylguanine bonding site, a CLIP-tag-O2-benzylcytosine bonding site, and a sortase-coupling bonding site.

In another example, the single template site includes a noncovalent template bonding site. In yet another example, the noncovalent template bonding site includes a polynucleotide hybridization site. In yet another example, the noncovalent template bonding site includes a noncovalent peptide binding site and the noncovalent peptide binding site is selected from a coiled-coil bonding site and an avidin-biotin bonding site.

In another example, the plurality of accessory sites for bonding accessory oligonucleotides to the scaffold include covalent accessory oligonucleotide bonding sites. In yet another example, the covalent accessory oligonucleotide bonding sites are selected from amine-NETS ester bonding sites, amine-imidoester bonding sites, amine-pentofluorophenyl ester bonding sites, amine-hydroxymethyl phosphine bonding sites, carboxyl-carbodiimide bonding sites, thiol-maleimide bonding sites, thiol-haloacetyl bonding sites, thiol-pyridyl disulfide bonding sites, thiol-thiosulfonate bonding sites, thiol-vinyl sulfone bonding sites, aldehyde-hydrazide bonding sites, aldehyde-alkoxyamine bonding sites, hydroxy-isocyanate bonding sites, azide-alkyne bonding sites, azide-phosphine bonding sites, transcyclooctene-tetrazine bonding sites, norbornene-tetrazine bonding sites, azide-cyclooctyne bonding sites, azide-norbornene bonding sites, oxime bonding sites, SpyTag-SpyCatcher bonding sites, Snap-tag-O⁶-Benzylguanine bonding sites, CLIP-tag-O2-benzylcytosine bonding sites, sortase-coupling bonding sites, and any combination of two or more of the foregoing.

In another example, the accessory oligonucleotide bonding sites include noncovalent accessory oligonucleotide bonding sites. In yet another example, the noncovalent accessory oligonucleotide bonding sites include polynucleotide hybridization sites. In still another example, the noncovalent accessory oligonucleotide bonding sites include noncovalent peptide binding sites and the noncovalent peptide binding sites are selected from one or both of coiled-coil bonding sites and avidin-biotin bonding sites.

In another example, the nanoparticle further includes a single template polynucleotide bonded to the single template site. In yet another example, the nanoparticle further includes a plurality of accessory oligonucleotides bonded to the plurality of accessory sites.

In another example, the nanoparticle is at least about 10 nm in diameter, at least about 20 nm in diameter, at least about 30 nm in diameter, at least about 40 nm in diameter, at least about 50 nm in diameter, at least about 60 nm in diameter, at least about 70 nm in diameter, at least about 80 nm in diameter, at least about 90 nm in diameter, at least about 100 nm in diameter, at least about 125 nm in diameter, at least about 150 nm in diameter, at least about 175 nm in diameter, at least about 200 nm in diameter, at least about 225 nm in diameter, at least about 250 nm in diameter, at least about 275 nm in diameter, at least about 300 nm in diameter, at least about 325 nm in diameter, at least about 350 nm in diameter, at least about 375 nm in diameter, at least about 400 nm in diameter, at least about 425 nm in diameter, at least about 450 nm in diameter, at least about 475 nm in diameter, at least about 500 nm in diameter, at least about 550 nm in diameter, at least about 600 nm in diameter, at least about 650 nm in diameter, at least about 700 nm in diameter, at least about 750 nm in diameter, at least about 800 nm in diameter, at least about 850 nm in diameter, at least about 900 nm in diameter, or at least about 950 nm in diameter.

In another aspect, provided is a method, including bonding a single template polynucleotide to the single template site of the nanoparticle.

In another aspect, provided is a method, including bonding a plurality of accessory oligonucleotides to the plurality of accessory sites of the nanoparticle.

In another aspect provided is a method, including at least one of bonding a single template polynucleotide to the single template site of the nanoparticle and bonding a plurality of accessory oligonucleotides to the plurality of accessory sites of the nanoparticle, further including synthesizing one or more scaffold-attached copies selected from copies of the template polynucleotide, copies of the polynucleotides complementary to the template polynucleotide, and copies of both, wherein the scaffold-attached copies extend from the accessory oligonucleotides.

In another example, the method further includes attaching the scaffold to a substrate, wherein attaching includes hybridizing accessory oligonucleotides with oligonucleotides attached to the substrate.

In yet another example of the method, the substrate includes a plurality of nanowells and the oligonucleotides attached to the substrate are attached within the plurality of nanowells. In yet a further example, no more than one scaffold binds within any one of the nanowells. In still another example, the method further includes synthesizing one or more substrate-attached copies selected from copies of the template polynucleotide, copies of the polynucleotides complementary to the template polynucleotide, and copies of both, wherein the substrate-attached copies extend from oligonucleotides attached to a substrate. In still a further example, the method further includes sequencing at least one of scaffold-attached copies and substrate-attached copies, wherein sequencing includes sequencing by synthesis.

### BRIEF DESCRIPTION OF THE DRAWINGS

These and other features, aspects, and advantages of the present disclosure will become better understood when the following detailed description is read with reference to the accompanying drawings, wherein:

FIG. 1 shows an example of a nanoparticle in accordance with aspects of the present disclosure.

FIGS. 2A-2F show of portions of an example of a scaffold of a nanoparticle including a DNA dendrimer in accordance with aspects of the present disclosure.

FIG. 3 shows examples of a nanoparticle including a single-stranded DNA scaffold in accordance with aspects of the present disclosure.

FIG. 4 shows an example of a polypeptide scaffold of a nanoparticle in accordance with aspects of the present disclosure.

FIG. 5 shows examples of attachment of a template to a nanoparticle in accordance with aspects of the present disclosure.

FIG. 6 shows an example of synthesizing a scaffold-attached copy of a template polynucleotide, in accordance with aspects of the present disclosure.

FIG. 7 shows an example of a covalent attachment of a polynucleotide, in accordance with aspects of the present disclosure.

FIG. 8 shows an example of a covalent attachment to amino acids of a scaffold, in accordance with the aspects of the present disclosure.

FIG. 9 shows an example of noncovalently attaching a template polynucleotide to a nanoparticle by hybridization, in accordance with aspects of the present disclosure.

FIG. 10 shows an example of noncovalently attaching a template polynucleotide to a nanoparticle by a coiled-coil peptide binding site, in accordance with aspects of the present disclosure.

FIG. 11 shows examples of a plurality of accessory sites of a scaffold of a nanoparticle for covalent attachment, in accordance with aspects of the present disclosure.

FIG. 12 shows an example of a nanoparticle in a nanowell, in accordance with aspects of the present disclosure.

FIG. 13 is a graph showing a number of nanoparticles per nanowell according to nanowell surface area, in accordance with aspects of the present invention.

FIGS. 14A-14D show an example of seeding a substrate with template polynucleotides using a DNA scaffold in accordance with aspects of the present disclosure.

### DETAILED DESCRIPTION

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

This disclosure relates to compositions and methods for increasing monoclonal clustering during SBS. In an example, principles of size exclusion are used to prevent individual template polynucleotides from seeding and therefore promoting clustering too close to each other. By associating each individual template polynucleotide with a nanoparticle of a given, sufficient spatial dimension, the template polynucleotides may be induced to attach to a substrate's surface sufficiently distal from each other to reduce formation of polyclonal clusters and increase formation of monoclonal clusters. A nanoparticle may include a bonding site for a template polynucleotide. The nanoparticle may have only one, single site for attachment of a template polynucleotide. One and only one template polynucleotide may therefore be capable of attaching to a nanoparticle, such that attachment of a template polynucleotide to the scaffold prevents attachment of a second template polynucleotide to the same nanoparticle, the attached template polynucleotide having occupied the single template polynucleotide bonding site thereof. Attachment of only a single template polynucleotide per nanoparticle, and resulting spatial distribution of template polynucleotides attached to such nanoparticles from each other due, directly or indirectly, to the sizes of the attached nanoparticles, reduces formation of polyclonal clusters.

The nanoparticle may also include other types of one or more bonding sites for attachment of the nanoparticle to compositions or surfaces in addition to a template polynucleotide, referred to herein as accessory bonding sites. For example, in addition to a single template polynucleotide bonding site, a nanoparticle may include accessory bonding sites that permit attachment of the nanoparticle to the surface of a substrate for us in an SBS process. In another example, a nanoparticle may possess one or more accessory bonding sites for attachment of one or more surface polymers to the nanoparticle. In another example, a nanoparticle may include one or more accessory bonding sites for attachment of an accessory oligonucleotide to the nanoparticle, wherein the oligonucleotide may bind to an end of a template polynucleotide or copy thereof as part of a clustering process, as described more fully below. In another example, such accessory oligonucleotides may be hybridizable to oligonucleotides attached to a surface of a substrate for use in an SBS process such that the nanoparticle with single template polynucleotide attached thereto may attach to such substrate surface.

Whereas a scaffold may include a single bonding site for a template polynucleotide and one or more accessory sites for attachment of, for example, an accessory oligonucleotide, the single template polynucleotide bonding site may be of a chemistry or structure different from that of accessory bonding sites. Of all of the bonding sites, the single template polynucleotide bonding site may be the only one having a chemistry or structure designed for attaching to a template polynucleotide with a corresponding chemistry or structure for attachment thereto. By comparison, the one or more accessory bonding sites may possess a different chemistry or structure, which is not compatible with binding or attaching to a template polynucleotide. Rather, the one or more accessory bonding sites may have a chemistry or structure compatible for binding or attaching to other compositions or structures to which the accessory bonding sites are intended to bind, such as accessory oligonucleotides, polymers, etc., and incompatible with binding or attaching to a template polynucleotide. Thus, a template polynucleotide would be incapable of binding or attaching to the one or more accessory bonding sites, resulting in attachment of only one template polynucleotide per nanoparticle, at the single template polynucleotide bonding site of the nanoparticle.

A template polynucleotide may be a polynucleotide obtained from a sample, such as a polydeoxyribonucleic acid isolated from a sample, or a cDNA molecule copied from a mRNA molecule that was obtained from a sample. An SBS process may be performed, for example, to determine a nucleotide sequence of a template polynucleotide, or to identify one or more polymorphisms or alterations in genetic sequence of a template polynucleotide in comparison to a reference sequence. A library may be prepared from one or more samples, the library including a plurality of template polynucleotides obtained from the one or more samples. Template polynucleotides may be obtained by obtaining polynucleotide sequences that are portions of sequences that were present in the sample or copied from the sample. By sequencing a plurality of template polynucleotides in an SBS process, sequence, genotype, or other sequence-related information may be determined as to the template polynucleotides and, when sequence information about a plurality of template polynucleotides in a library is collected and analyzed, about the sample from which the library was obtained.

A template polynucleotide may be processed as part of a process of obtaining a template polynucleotide from sample. Part of processing may include adding polynucleotide sequences, such as to the 5-prime, 3-prime, or both ends of the template to assist in subsequence SBS processing. As further disclosed herein, a template polynucleotide may further be modified by adding features that promote or permit forming a bond with a site on a nanoparticle.

A template polynucleotide may be of any given length suitable for obtaining sequencing information in an SBS process. For example, a template polynucleotide may be about 50 nucleotides in length, about 75 nucleotides in length, about 100 nucleotides in length, about 125 nucleotides in length, about 150 nucleotides in length, about 175 nucleotides in length, about 200 nucleotides in length nucleotides in length, about 225 nucleotides in length, about 250 nucleotides in length, about 275 nucleotides in length, about 300 nucleotides in length, about 325 nucleotides in length, about 350 nucleotides in length, about 375 nucleotides in length, about 400 nucleotides in length, about 425 nucleotides in length, about 450 nucleotides in length, about 475 nucleotides in length, about 500 nucleotides in length, about 525 nucleotides in length, about 550 nucleotides in length, about 575 nucleotides in length, about 600 nucleotides in length, about 625 nucleotides in length, about 650 nucleotides in length, about 675 nucleotides in length, about 700 nucleotides in length, about 725 nucleotides in length, about 750 nucleotides in length, about 775 nucleotides in length, about 800 nucleotides in length, about 825 nucleotides in length, about 850 nucleotides in length, about 875 nucleotides in length, about 900 nucleotides in length, about 925 nucleotides in length, about 950 nucleotides in length, about 975 nucleotides in length, about 1,000 nucleotides in length, about 1,025 nucleotides in length, about 1,050 nucleotides in length, about 1,075 nucleotides in length, about 1,100 nucleotides in length, about, 1,125 nucleotides in length, about 1,150 nucleotides in length, about 1,175 nucleotides in length, about 1,200 nucleotides in length, about 1,225 nucleotides in length, about 1,250 nucleotides in length, about 1,275 nucleotides in length, about 1,300 nucleotides in length, about 1,325 nucleotides in length, about 1,350 nucleotides in length, about 1,375 nucleotides in length, about 1,400 nucleotides in length, about 1,425 nucleotides in length, about 1,450 nucleotides in length, about 1,475 nucleotides in length, about 1,500 nucleotides in length, or longer.

In some examples, there may be two or more different populations of accessory bonding sites on a nanoparticle, some with one type of chemistry or structure compatible with binding or attaching to one population of compositions or structures, and others with a second type of chemistry or structure compatible with binding or attaching to another population of compositions or structures. For example, one population of accessory sites may have a chemistry or structure compatible with binding to accessory oligonucleotides which accessory oligonucleotides may bind to copies of template polynucleotides that participate in, for example, clustering of a template polynucleotide on a nanoparticle, as described more fully below, while other accessory sites may have a different chemistry or structure compatible with binding or attaching to a surface of a substrate for performing SBS.

A nanoparticle may include a scaffold. A scaffold is a structural component of a nanoparticle occupying volume according to a minimum amount of distance desired between template nanoparticles or a maximum density of template nanoparticles attached to nanoparticles as may be desirable for a given application. A scaffold may include the aforementioned bonding sites, as in single template polynucleotide binding site and one or more accessory bonding site. Together, the scaffold with bonding sites, may constitute a nanoparticle. A scaffold may be synthesized so as to include, and may include once synthesized, more than one type of chemistry or structure for attachment. That is, it may be synthesized to include or be modified to include a single site of attachment to a template polynucleotide, plus one or more additional bonding sites with a different chemistry or structure from the single template polynucleotide bonding site corresponding to accessory bonding sites.

A scaffold may by synthesized from several different substituent components. In an example, a scaffold may be synthesized from one or more scaffold deoxyribonucleic acid (DNA) molecules. DNA molecules may be designed and structured as further disclosed herein so as to permit inclusion of different bonding sites (i.e., for a template polynucleotide as well as accessory binding sites) and also to provide size-exclusion properties for distancing template polynucleotides from each other once attached to a polynucleotide. In some examples, a scaffold may include a plurality of DNA molecules hybridized together so as to form a dendrimer. For example, adapters may be formed including a plurality of, such as three, strands of DNA, or oligodeoxyribonucleotide (oligo-DNA) molecules that can hybridize to each other by Watson-Crick base pairing so as to form a Y-shape, with one end of each hybridizing to one of the other two and the other end of each hybridizing to the other of the other two.

Such adapters may form a constitutional repeating until of a dendrimer. For example, each end of the Y-shaped adapter may have an overhang of DNA, where the end of one of the oligo-DNAs extends beyond the portion of which hybridizes to any other oligo-DNA. An adapter of one generation of such dendrimer may have an overhang on one end of the Y-shape, referred to here as the upstream end, that can hybridize with an overhang of an and of another Y adapter that constitutes a constitutional repeating unit of an immediately preceding generation of the dendrimer. And the other two ends of the adapter, referred to as the downstream ends, may each have an overhang that can hybridize with an overhang of an upstream end of a Y adapter that constitutes a constitutional repeating unit of an immediately following generation of the dendrimer. Thus, an adapter of one generation may attach to two adapters in the next generation, which may attach to four adapters of the following generation, which may attach to eight adapters of the following generation, and so on. Any one end of one of the terminal Y adapters, whether a downstream overhang of any generation, such as the last generation, not hybridized to an upstream overhang of another adapter, or the upstream overhang of the first generation, may include or be attached to the single template polynucleotide binding site. In an example, the DNA-oligo including the upstream overhang of the first generation adapted may itself be an extension of a template polynucleotide, added thereto during sample preparation. Other ends or overhangs may include or be attached to accessory sites.

In other examples, a scaffold may include one or more single-stranded DNA (ssDNA) molecules modified or structured so as to permit attachment thereto of a single template polynucleotide and one or more accessory compositions or a structure, to one or more accessory bonding site. Various methods for producing an ssDNA-based scaffold may be used. In an example, a double-stranded closed loop or plasmid may serve as a coding sequence for an ssDNA scaffold molecule, in a rolling circle amplification process. Replication of a strand thereof by a strand-displacing DNA polymerase (e.g., Phi29) may produce an ssDNA molecule including concatemerized copies of the copied strand of the circular coding strand. Reaction conditions may be adopted so as to result in synthesis of an ssDNA scaffold of a desired size. A 5-prime or 3-prime end may be further modified to include or be attached or attachable to a single template polynucleotide molecule, as the single template site. Accessory sites may include the other end of the ssDNA scaffold molecule, or modifications to or of individual nucleotides of the strand as further described below.

In another example, an ssDNA scaffold may be synthesized by use of a template-independent polymerase (e.g., terminal deoxynucleotidyl transferase, or TdT). TdT incorporates deoxynucleotides at the 3-prime-hydroxyl terminus of a single-stranded DNA strand, without requiring or copying a template. A 5-prime or 3-prime end may be further modified to include or be attached or attachable to a single template polynucleotide molecule, as the single template site. Accessory sites may include the other end of the ssDNA scaffold molecule, or modifications to or of individual nucleotides of the strand as further described below. As used herein, a “nucleotide” includes a nitrogen-containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine.

In another example, an ssDNA scaffold may be synthesized by producing a plurality of single-stranded DNA molecules by any applicable method and ligating them together to form a single ssDNA molecule as a scaffold. For example, a polymerase may polymerize formation of a nascent strand of DNA by copying a linearized DNA coding strand, in a run-off polymerization reaction (i.e., where the polymerase ceases extending a nascent strand upon reaching a 5-prime end of a coding strand). A plurality of ssDNA products may be synthesized, then ligated end-to-end for formation of a single ssDNA scaffold. In an example, ligation of one ssDNA product to another may be accomplished with the aid of a splint. For example, a short oligo-DNA may be designed whose 3-prime end is complementary of the 5-prime end of one ssDNA product and whose 5-prime end is complementary to the 3-prime end of another ssDNA product, such that hybridization of the DNA-oligo to the two ssDNA products brings the 5-prime end of one together with the 3-prime end of the other in a nicked, double-stranded structure where they meet hybridized to the DNA-oligo. A DNA ligase (e.g., T4) may then be used to enzymatically ligate the two ends together to form a single ssDNA molecule from the two. Additional reactions may be included with DNA-oligos for splint-aided ligation of one or both ends of the product of such first reaction to another ssDNA product, and so on, for construction of an ssDNA scaffold as may be desired.

A template polynucleotide for attachment to a scaffold may be of any suitable length, including for sequencing in an SBS process. For example, a template polynucleotide may be about 50 nucleotides in length, about 75 nucleotides in length, about 100 nucleotides in length, about 125 nucleotides in length, about 150 nucleotides in length, about 175 nucleotides in length, about 200 nucleotides in length, about 225 nucleotides in length, about 250 nucleotides in length, about 275 nucleotides in length, about 300 nucleotides in length, about 325 nucleotides in length, about 350 nucleotides in length, about 375 nucleotides in length, about 400 nucleotides in length, about 425 nucleotides in length, about 450 nucleotides in length, about 475 nucleotides in length, about 500 nucleotides in length, about 525 nucleotides in length, about 550 nucleotides in length, about 575 nucleotides in length, about 600 nucleotides in length, about 625 nucleotides in length, about 650 nucleotides in length, about 675 nucleotides in length, about 700 nucleotides in length, about 725 nucleotides in length, about 750 nucleotides in length, about 775 nucleotides in length, about 800 nucleotides in length, about 825 nucleotides in length, about 850 nucleotides in length, about 875 nucleotides in length, about 900 nucleotides in length, about 925 nucleotides in length, about 950 nucleotides in length, about 975 nucleotides in length, about 1,000 nucleotides in length, about 1,100 nucleotides in length, about 1,200 nucleotides in length, about 1,300 nucleotides in length, about 1,40 nucleotides in length, about 1,500 nucleotides in length, about 1,600 nucleotides in length, about 1,700 nucleotides in length, about, 1,800 nucleotides in length, about 1,900 nucleotides in length, about 2,000 nucleotides in length, or longer.

Attachment of a single template polynucleotide or accessory (e.g., accessory oligonucleotide, accessory composition, or accessory structure) to a DNA scaffold may be accomplished by inclusion of moieties or structures on the scaffold and template polynucleotide or accessory that are complementary to each other, meaning they are configured to bind to one another, covalently or non-covalently, to form an attachment therebetween. They may be complementary for covalent binding or complementary for non-covalent binding. A DNA scaffold may include a single template site with a moiety or structure that is complementary to or with a moiety or structure (a single template site) that is attached to a template polynucleotide. The DNA scaffold may also include or be attached to other moieties or structures that are complementary to or with a moiety or structure (accessory sites) attached to an accessory. Cross-reactivity between a moiety or structure attached to a template polynucleotide and a moiety or structure of an accessory site should be avoided, to prevent attachment of more than one template polynucleotide to a DNA scaffold. Cross-reactivity between a moiety or structure attached to an accessory and a moiety or structure of the single template site should also be avoided, to prevent occupation of the single template site by accessories that prevents attachment of a single template polynucleotide thereto. In some examples, such cross-reactivity may be avoided by blocking the single template site or accessory sites chemically while accessories bind to the accessory sites or single template polynucleotides attach to the single template site, respectively, then unblocking the unoccupied site to permit attachment of the single template polynucleotide or accessory thereto.

A non-exclusive list of complementary binding partners is presented in Table 1:

| | Example moiety/structure on | Example moiety/structure on |
|---|---|---|
| | (a) scaffold-attached bonding | (a) template polynucleotide or |
| | site or (b) template | accessory or (b) scaffold- |
| Bonding site | polynucleotide or accessory | attached bonding site |
| amine-NHS | amine group, —NH₂ | 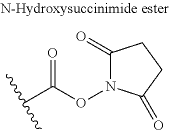 [SMILES: *C(=O)ON1C(=O)CCC1=O] |
| amine-imidoester | amine group, —NH₂ | 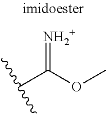 [SMILES: *C(=[NH2+])OC] |
| amine-pentofluorophenyl ester | amine group, —NH₂ | 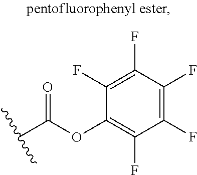 [SMILES: *C(=O)Oc1c(F)c(F)c(F)c(F)c1F] |
| amine-hydroxymethyl phosphine | amine group, —NH₂ | 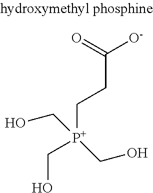 [SMILES: O=C([O-])CC[P+](CO)(CO)CO] |
| amine-carboxylic acid | amine group, —NH₂ | carboxylic acid group, |
| | | —C(═O)OH (e.g., following |
| | | activation of the carboxylic |
| | | acid by a carbodiimide such as |
| | | EDC (1-ethyl-3-(-3- |
| | | dimethylaminopropyl) |
| | | carbodiimide hydrochloride) or |
| | | DCC (N′,N′-dicyclohexyl |
| | | carbodiimide) to allow for |
| | | formation of an amide bond |
| | | of the activated carboxylic |
| | | acid with an amine group) |
| thiol-maleimide | thiol, —SH | 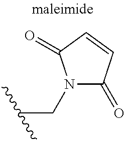 [SMILES: *CN1C(=O)C=CC1=O] |
| thiol-haloacetyl | thiol, —SH | 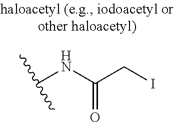 [SMILES: *NC(=O)CI] |
| thiol-pyridyl disulfide | thiol, —SH | 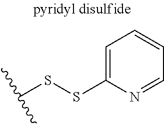 [SMILES: *SSc1ccccn1] |
| thiol-thiosulfonate | thiol, —SH | 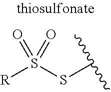 |
| thiol-vinyl sulfone | thiol, —SH | 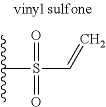 [SMILES: *S(=O)(=O)C=C] |
| aldehyde-hydrazide | aldehyde, —C(═O)H | 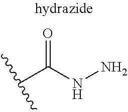 [SMILES: *C(=O)NN] |
| aldehyde-alkoxyamine | aldehyde, —C(═O)H | 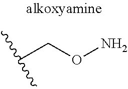 [SMILES: *CON] |
| hydroxy-isocyanate | hydroxyl, —OH | 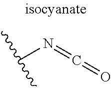 [SMILES: *N=C=O] |
| azide-alkyne | azide, —N₃ | 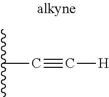 [SMILES: *C#C] |
| azide-phosphine | azide, —N₃ | 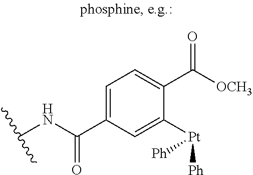 |
| azide-cyclooctyne | azide, —N₃ | 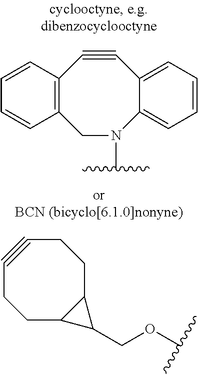 [SMILES: *N1Cc2ccccc2C#Cc2ccccc21 ; *OCC1C2CCC#CCCC21] |
| azide-norbornene | azine, —N₃ | 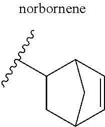 [SMILES: *C1CC2C=CC1C2] |
| transcyclooctene-tetrazine | 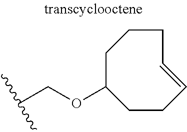 [SMILES: *COC1CC/C=C/CCC1] | 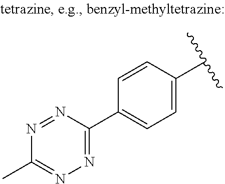 [SMILES: *c1ccc(-c2nnc(C)nn2)cc1] |
| norbornene-tetrazine | 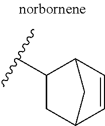 [SMILES: *C1CC2C=CC1C2] | 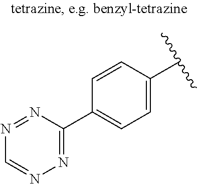 [SMILES: *c1ccc(-c2nncnn2)cc1] |
| oxime | aldehyde or ketone (e.g., amine | alkoxyamine |
| | group or N-terminus of | |
| | polypeptide converted to an | |
| | aldehyde or ketone by | |
| | pyroxidal phosphate) | |
| SpyTag-SpyCatcher | SpyTag: amino acid sequence | SpyCatcher amino acid |
| | AHIVMVDAYKPTK | sequence: |
| | (SEQ ID NO: 1) | MKGSSHHHHHHVDIPTT |
| | | ENLYFQGAMVDTLSGLS |
| | | SEQGQSGDMTIEEDSATH |
| | | IKFSKRDEDGKELAGAT |
| | | MELRDSSGKTISTWISDG |
| | | QVKDFYLYPGKYTFVET |
| | | AAPDGYEVATAITFTVNE |
| | | QGQVTVNGKATK |
| | | (SEQ ID NO: 2) |
| SNAP-tag-O⁶- Benzylguanine | SNAP-tag (O-6- methylguanine-DNA methyltransferase) | 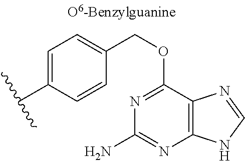 [SMILES: *c1ccc(COc2nc(N)nc3[nH]cnc23)cc1] |
| CLIP-tag-O²- benzylcytosine | CLIP-tag (modified O-6- methylguanine-DNA methyltransferase) | 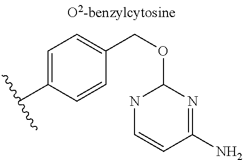 [SMILES: *c1ccc(COC2N=C(N)C=CN2)cc1] |
| Sortase-coupling | -Leu-Pro-X-Thr-Gly | -G1y₍₃₋₅₎ |

Any of the foregoing can be added to or included in a scaffold as disclosed herein for attachment to a template polynucleotide or accessories such as accessory oligonucleotides, which template polynucleotide or accessory may include or be modified to include a complementary moiety or structure of the foregoing pairs for bonding to the scaffold.

Any suitable bioconjugation methods for adding or forming bonds between such pairs of complementary moieties or structures may be used. Modified nucleotides may be commercially available possessing examples of one or the other of examples of such pairs of complementary moieties or structures, and methods for including one or more of such examples of moieties or structures in or attaching or including them to polymer, a nucleotide, or polynucleotide are also known. Also commercially available may be e bifunctional linker molecules with a moiety or structure from one complementary pair of bonding partners listed in Table 1 at one end and a moiety or structure from another complementary pair of bonding partners listed in Table 1. A moiety or structure of a scaffold, template polynucleotide, or of an accessory, or an oligo or polypeptide being attached to any of the foregoing features to as to provide a moiety or structure for bonding between any of such foregoing features, may be bound to one end of such a linker, resulting in the initial moiety or structure being effectively replaced with another, i.e., the moiety or structure present on the other end of the linker.

For example, a bifunctional linker may have on one end a moiety from among those listed in Table 1, such as an NETS-ester group. At the other end it may have another group, such as an azide group. The ends may be connected to each other by a linker, such as, for example, one or more PEG groups, alkyl chain, combinations thereof in a linking sequence, etc. If a bonding site (such as of a scaffold, or of a template polynucleotide or an accessory) has an amine group for bonding, the NETS-ester end of the bifunctional linker can be bound to the amine group, leaving the free azide end available for bonding to a composition (e.g., a template polynucleotide or an accessory, or a scaffold) bearing a bonding partner for an azide group (e.g., alkyne, phosphine, cyclooctyne, or norbornene). Or, if a bonding site (such as of a scaffold, or of a template polynucleotide or an accessory) has bonding partner for an azide group (e.g., alkyne, phosphine, cyclooctyne, or norbornene), the azide end of the bifunctional linker can be bound to the amine group, leaving the free NETS-ester end available for bonding to a composition (e.g., a template polynucleotide or an accessory, or a scaffold) bearing an amine group. Many other examples of bifunctional linkers are commercially available including on an end a moiety identified in Table 1 for forming one type of bonding site and on the other end a different moiety identified in Table 1 for forming another type of bonding site.

Modified amino acids may be commercially available possessing examples of one or the other of examples of such pairs of complementary moieties or structures, and methods for including one or more of such examples of moieties or structures in or attaching them to an amino acid or polypeptide are also known. Methods for forming bonds between members of such pairs of complementary moieties or structures are known. Thus, such complementary moieties or structures can be added to or included in a scaffold and a template polynucleotide or a scaffold and an accessory to form bonding sites and permit attachment therebetween.

In an example, a single template polynucleotide bonding site of a scaffold may include a first moiety or structure from Table 1 and one or more accessory sites a scaffold may include one or more other moieties or structures from Table 1. The first moiety or structure may be able to form a bonding site with a first bonding partner and the other moieties or structures may be able to form bonding sites with another bonding partner or partners, under conditions wherein the first bonding partner will not react with the other bonding partner or partners to form a bonding site, and the other moieties or structures will not react with the first bonding partner to form a bonding site. In another example, the first moiety or structure and the other moieties or structures are selected such that they would not form bonding partners with each other.

As used herein, the term “polypeptide” is intended to mean a chain of amino acids bound together by peptide bonds. The terms “protein” and “polypeptide” may be used interchangeably. A polypeptide may include a sequence of a number of amino acids bound to each other by peptide bonds and the number of amino acids may be about 2 or more, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 110 or more, about 120 or more, about 130 or more, about 140 or more, about 150 or more, about 160 or more, about 170 or more, about 180 or more, about 190 or more, about 200 or more, about 225 or more, about 250 or more, about 275 or more, about 300 or more, about 325 or more, about 350 or more, about 375 or more, about 400 or more, about 425 or more, about 450 or more, about 475 or more, about 500 or more, about 550 or more, about 600 or more, about 650 or more, about 700 or more, about 750 or more, about 800 or more, about 850 or more, about 900 or more, about 950 or more, about 1000, or higher.

In some cases, a polypeptide, or protein, may adopt a structure or three-dimensional conformation to promote or permit bonding to another bonding partner such as another polypeptide that also adopts a three-dimensional structure conducive to such bonding, or other, non-protein bonding partners. A polypeptide may also adopt a three-dimensional conformation conducive to performing enzymatic reactions on other substrate polypeptides or other molecules, or so as to serve as a substrate for another enzymatic or other reaction. A polypeptide may also adopt a three-dimensional conformation such that a site or sites, such as an amino terminal, a carboxyl terminal, a side group of an amino acid, or a modification to an amino acid, may be accessible for bonding with another molecule.

Various bioconjugation chemistries can be used for attaching a template polynucleotide to a nucleotide of a DNA scaffold, or to a 5-prime or 3-prime (e.g., a nucleotide included in an unhybridized overhang or other nucleotide of a dendrimer DNA scaffold, or 3-prime or 5-prime terminal or nucleotide therebetween of an ssDNA scaffold). Furthermore, modifications to a nucleotide included in a DNA scaffold, such as on a phosphate group, the base, or the sugar, may be implemented to provide a single template site for attachment. A chemical moiety may be included in or added to such a site having an ability to form a covalent conjugation to a complementary chemical moiety, which complementary moiety may be attached to or included in a template polynucleotide. A template polynucleotide may then be conjugated to the DNA scaffold, such as through covalent attachment between the complementary chemical moieties. In an example, nucleotides modified to include an attachment moiety, capable of being incorporated into a polynucleotide strand by a polymerase but also including a chemical moiety with which a complementary moiety may react to form a covalent bond therebetween, may be included during a polymerization reaction to form a DNA scaffold or part thereof.

In another example, a DNA scaffold may include or be attached to, as a single template site, a polypeptide sequence capable of forming a covalent attachment to another polypeptide sequence or other chemical moiety. Such other polypeptide or other chemical moiety may then be included in or attached to a template polynucleotide, such that the single template site of the scaffold and the template polynucleotide may covalently bond to each other. Alternatively, the template polynucleotide may have the first such polypeptide sequence, and the single template site of the scaffold may have such other polypeptide sequence or other chemical moiety capable of covalently bonding to the polypeptide sequence of the template polynucleotide. Non-limiting examples of such pairs include the SpyTag/SpyCatcher system, the Snap-tag/O⁶-Benzylguanine system, and the CLIP-tag/O²-benzylcytosine system.

Amino acid sequences for the complementary pairs of the SpyTag/SpyCatcher system and polynucleotides encoding them may be available. Examples of sequences are provided in Table 1. Several amino acid site mutations for a SpyTag sequence and for a SpyCatcher sequence may be available for inclusion in recombinant polypeptides. A Snap-tag is a functional O-6-methylguanine-DNA methyltransferase, and a CLIP-tag is a modified version of Snap-tag. Nucleotide sequences encoding Snap-tag, CLIP-tag, SpyCatcher, may be commercially available for subcloning and inclusion in engineered polypeptide sequences.

Alternatively, complementary pairs for covalent attachment on a single template site of a scaffold and a template polynucleotide may be covalently attached to each other via an enzymatically catalyzed formation of a covalent bond. For example, a single template site of a scaffold and a template polynucleotide may include motifs capable of covalent attachment to each other by sortase-mediated coupling, e.g. a LPXTG amino acid sequence on one and an oligoglycine nucleophilic sequence on the other (with a repeat of, e.g., from 3 to 5 glycines). Sortase-mediated transpeptidation may then be carried out to result in covalent attachment of the scaffold and template polynucleotide at the single template site.

In another example, a DNA scaffold may include a region for non-covalent attachment of a single template polynucleotide at a single template site. For example, an unhybridized overhang of a dendrimer DNA scaffold may be hybridizable by Watson-Crick base pairing to an end of a template polynucleotide. In an example, the upstream overhang of the adapter of the first generation of the dendrimer may be include a nucleotide sequence complementary to a nucleotide sequence included in an end of a template polynucleotide. Or a 3-prime or 5-prime end of an ssDNA scaffold may have a nucleotide sequence complementary to a nucleotide sequence included in an end of a template polynucleotide. Hybridization of such complementary nucleotide sequences to each other through Watson-Crick base-pairing may accordingly permit non-covalent attachment of the single template site of the DNA scaffold to a template polynucleotide.

In another example, a DNA scaffold and template polynucleotide may include or be attached to complementary peptide binding sites. For example, the DNA scaffold and template polynucleotide may include or be attached to peptide sequences that may bind to each other as complementary pairs of a coiled coil motif. A coiled coil motif is a structural feature of some polypeptides where two or more polypeptide strands each form an alpha-helix secondary structure and the alpha-helices coil together to form a tight non-covalent bond. A coiled coil sequence may include a heptad repeat, a repeating pattern of the seven amino acids HPPHCPC (where H indicates a hydrophobic amino acid, C typically represents a charged amino acid and P represents a polar, hydrophilic amino acid). An example of a heptad repeat is found in a leucine zipper coiled coil, in which the fourth amino acid of the heptad is frequently leucine.

A DNA scaffold may include or be attached to one amino acid sequence that forms part of a coiled coil bonding pair and a template polynucleotide may be attached to another amino acid sequence that forms another part of a coiled coil bonding pair, complementary to that which is or is attached to the DNA scaffold, such that the two attach to each other. For example, a DNA scaffold may be covalently attached to one amino acid sequence that forms part of a coiled coil bonding pair and a template polynucleotide may be attached to another amino acid sequence that forms another part of a coiled coil bonding pair, complementary to that which is or is attached to the DNA scaffold, such that the two attach to each other.

In another example, the DNA scaffold and the template polynucleotide may each include or be attached to other complementary partners of peptide pairs that bind together non-covalently. An example includes a biotin-avidin binding pair. Biotin and avidin peptides (such as avidin, streptavidin, and neutravidin, all of which are referred to collectively as “avidin” herein unless specifically stated otherwise) form strong noncovalent bonds to each other. One part of such pair, whether binding portion of biotin or of avidin, may be part of or attached to either the DNA scaffold or template polynucleotide, with the complementary part correspondingly part of or attached to the DNA scaffold or template polynucleotide, permitting non-covalent attachment therebetween.

Numerous methods are available for including one or more biotin moiety in or adding one or more biotin moiety to a DNA molecule, template polynucleotide, DNA scaffold, oligo-DNA, polypeptide scaffold, other polypeptide, or other composition for bonding molecules together as disclosed herein (such as template polynucleotides to a scaffold, or accessories to a scaffold). For example, biotinylated nucleotides are commercially available for incorporation into a DNA molecule by a polymerase, and kits are commercially available for adding a biotin moiety to a polynucleotide or a polypeptide. Biotin residues can also be added to amino acids or modified amino acids or nucleotides or modified nucleotides. Linking chemistries shown in Table 1 can also be used for adding a biotin group to proteins such as on carboxylic acid groups, amine groups, or thiol groups. Several biotin ligase enzymes are also available for enzymatically targeted biotinylation such as of polypeptides (e.g., of the lysine reside of the AviTag amino acid sequence GLNDIFEAQKIEWHE (SEQ ID NO:3) included in a polypeptide). A genetically engineered ascorbate peroxidase (APEX) is also available for modifying biotin to permit biotinylation of electron-rich amino acids such as tyrosine, and possibly tryptophan, cysteine, or histidine.

In another example, a polypeptide including the amino acid sequence DSLEFIASKLA (SEQ ID NO:4) may be biotinylated (at the more N-terminal of the two S residues present in the sequence), which is a substrate for Sfp phosphopantetheinyl transferase-catalyzed covalent attachment thereto with small molecules conjugated to coenzyme A (CoA). For example, a polypeptide including this sequence could be biotinylated through covalent attachment thereto by a CoA-biotin conjugate. This system may also be used for attaching many other types of bonding moieties or structures identified in Table 1 for use in creating bonding sites for a scaffold to bond to a DNA molecule or polypeptide or other molecule as disclosed herein. For example, a CoA conjugated to any of the reactive pair moieties identified in Table 1 could be covalently attached to a polypeptide containing the above-identified sequence by Sfp phosphopantetheinyl transferase, thereby permitting bonding of another composition thereto that includes the complementary bonding partner.

Other enzymes may be used for adding bonding moiety to a polypeptide. For example, a lipoic acid ligase enzyme can add a lipoic acid molecule, or a modified lipoic acid molecule including a bonding moiety identified in Table 1 such as an alkyne or azide group, can be covalently linked to the amine of a side group of a lysine reside in an amino acid sequence DEVLVEIETDKAVLEVPGGEEE (SEQ ID NO:5) or GFEIDKVWYDLDA (SEQ ID NO:6) included in a polypeptide. In another example, a scaffold, template polynucleotide, or other polypeptide or DNA molecule included therein or intended to be bonded thereto may include or be attached to an active serine hydrolase enzyme. Fluorophosphonate molecules become covalently linked to serine residues in the active site of serine hydrolase enzymes. Commercially available analogs of fluorophosphonate molecules including bonding moieties identified in Table 1, such as an azide group or a desthiobiotin group (an analog of biotin that can bind to avidin). Thus, such groups can be covalently attached to serine hydrolase enzyme included in or attached to a polypeptide or DNA molecule used in or attached to a scaffold as disclosed herein and such bonding moiety or structure can be covalently added thereto by use by attachment of a suitable modified fluorophosphonate molecule for creating a bonding site on such protein for a complementary bonding partner from Table 1 (such as for azide-alkyne, azide-phosphine, azide-cyclooctyne, azide-norbornene, or desthiobiotin-avidin bonding).

Any of the foregoing methods of biotinylating compositions to promote bonding to a polypeptide including an avidin sequence (such as an avidin polypeptide included in or attached to another composition), or otherwise adding functional groups to polypeptides, as part of a scaffold, attached to a scaffold, part of an accessory, or attached to an accessory or template polynucleotide, for bonding between a scaffold and a template polynucleotide or between a scaffold and an accessory, may be used for permitting or promoting bonding between such components as disclosed herein.

In another example, a scaffold may be synthesized of amino acids, such as a polypeptide or protein molecule. In an example, a single template site for attachment of a template polynucleotide may be or be attached to an N-terminus or a C-terminus of such polypeptide scaffold. In another example, a single template site for attachment of a template polynucleotide may be or be attached to an internal amino acid of the polypeptide scaffold. Various bioconjugation chemistries can be used for attaching a template polynucleotide to a side group of an amino acid between the C- and N-termini of the polypeptide, for example, or to one of the termini. Furthermore, modifications to an amino acid of the polypeptide scaffold, such as to a side chain of one of the amino acids, may be implemented to provide a single template site for attachment. A chemical moiety may be included in or added to such a site having an ability to form a covalent conjugation to a complementary chemical moiety, which complementary moiety may be attached to or included in a template polynucleotide. A template polynucleotide may then be conjugated to the polypeptide scaffold, such as through covalent attachment between the complementary chemical moieties.

In another example, a polypeptide scaffold may include or be attached to, as a single template site, a polypeptide sequence capable of forming a covalent attachment to another polypeptide sequence or other chemical moiety. Such other polypeptide or other chemical moiety may then be included in or attached to a template polynucleotide, such that the single template site of the scaffold and the template polynucleotide may covalently bond to each other. Alternatively, the template polynucleotide may have the first such polypeptide sequence, and the single template site of the scaffold may have such other polypeptide sequence or other chemical moiety capable of covalently bonding to the polypeptide sequence of the template polynucleotide. Non-limiting examples of such pairs include the SpyTag/SpyCatcher system, the Snap-tag/O⁶-Benzylguanine system, and the CLIP-tag/O²-benzylcytosine system. Alternatively, complementary pairs for covalent attachment on a single template site of a scaffold and a template polynucleotide may be covalently attached to each other via an enzymatically catalyzed formation of a covalent bond. For example, a single template site of a scaffold and a template polynucleotide may include motifs capable of covalent attachment to each other by sortase-mediated coupling, e.g. a LPXTG amino acid sequence on one and an oligoglycine nucleophilic sequence on the other (with a repeat of, e.g., from 3 to 5 glycines). Sortase-mediated transpeptidation may then be carried out to result in covalent attachment of the scaffold and template polynucleotide at the single template site.

In another example, a polypeptide scaffold may include a region for non-covalent attachment of a single template polynucleotide at a single template site. For example, an oligo-DNA may be covalently attached to a single site on the polypeptide scaffold. For example, complementary chemical moieties on the polypeptide scaffold and the oligo-DNA may permit covalent attachment between them much as described above for direct covalent attachment of a template polynucleotide and a polypeptide scaffold. The oligo-DNA may have a nucleotide sequence complementary to part of a template polynucleotide, such as to 3-prime or 5-prime end of a template polynucleotide. Complementarity between such oligo-DNA and template polynucleotide may permit, through Watson-Crick base-pairing, hybridization between a portion of the template oligonucleotide and the oligo-DNA attached to the polypeptide scaffold.

In another example, a polypeptide scaffold and template polynucleotide may include or be attached to complementary peptide binding sites. For example, the peptide scaffold and template polynucleotide may include or be attached to peptide sequences that may bind to each other as complementary pairs of a coiled coil motif. A polypeptide scaffold may include or be attached to one amino acid sequence that forms part of a coiled coil bonding pair and a template polynucleotide may be attached to another amino acid sequence that forms another part of a coiled coil bonding pair, complementary to that which is or is attached to the polypeptide scaffold, such that the two attach to each other.

In another example, a polypeptide scaffold and the template polynucleotide may each include or be attached to other complementary partners of peptide pairs that bind together non-covalently. An example includes a biotin-avidin binding pair. Biotin and avidin peptides form strong noncovalent bonds to each other. One part of such pair, whether a binding portion of biotin or of avidin, may be part of or attached to either the polypeptide scaffold or template polynucleotide, with the complementary part correspondingly part of or attached to the polypeptide scaffold or template polynucleotide, permitting non-covalent attachment therebetween.

For attachment to a single template site of a DNA scaffold or of a polypeptide scaffold, a template polynucleotide may have a complementary attachment moiety or structure added thereto. In an example, during preparation of a library sample, a plurality of template polynucleotides may be prepared for sequencing. Commonly during such sample preparation, template polynucleotides of the library sample are modified to include particular nucleotide sequences in addition to the sequences already included therein as part of the library to be sequenced. Such added nucleotide sequences may serve any of various functions, including for subsequent identification of the template polynucleotide or attachment to a surface of an SBS substrate as part of a seeding process. In accordance with the present disclosure, such preparation of template polynucleotides may also include a complementary attachment moiety or structure being attached thereto or included therein.

For example, for a dendrimer DNA scaffold, preparation of a template polynucleotide may include adding to or including in the template polynucleotide an oligonucleotide with a nucleotide sequence corresponding to the nucleotide sequence of the upstream end of the adapter of the first generation of the dendrimer. The first generation adapter may then include, as one of the three polynucleotide sequences of which it is constituted, such sequence as was added to the template polynucleotide. In another example, a nucleotide sequence may be added to a template polynucleotide complementary to an overhang of an adapter of a dendrimer DNA scaffold, such as the upstream overhang of the adapter of the first generation of the dendrimer DNA scaffold.

In another example, preparation of a template polynucleotide may include attachment of a nucleotide sequence in the template polynucleotide, such as extending from one of its ends, and the sequence is complementary to another sequence which other sequence is included in or attached to the single template site of the scaffold. Hybridization due to Watson-Crick base pairing results in bonding between the two. In another example, an accessory, such as an accessory oligonucleotide, may be modified to permit covalent attachment to it of a moiety or structure that is complementary thereto. For example, modifications to a nucleotide included in an accessory such as an accessory oligonucleotide, such as on a phosphate group, the base, or the sugar, may be included to provide a site for covalent attachment to accessory sites of a scaffold. Accessory sites of the scaffold may in turn include a complementary moiety or structure permitting attachment to accessories such as oligo-DNA accessories. In an example, nucleotides modified to include an attachment moiety with which a complementary moiety of an accessory bonding site of a scaffold, included in a polynucleotide sequence added to a template polynucleotide during sample preparation. Numerous modified nucleotides bearing such chemical moieties are commercially available for covalent attachment of compositions to DNA molecules in which such modified nucleotides have been incorporated.

In another example, a template polynucleotide may be modified, such as during sample preparation, by attaching to it a polypeptide. Such polypeptide may possess an amino acid sequence and structure so as to be complementary to an amino acid structure of a single template site of a scaffold, such that the template polynucleotide may attach, via its attached polypeptide, to the single template site of the scaffold. Examples of pairs of polypeptides for covalent or noncovalent bonding between a single template site of a scaffold and a template polynucleotide were provided above and include, as non-limiting examples, alpha-helical amino acid sequences with heptad repeats for formation of coiled coil attachments to one another, biotin-avidin binding pairs, SpyTag/SpyCatcher system, LPXTG/oligoglycine nucleophilic pairs for sortase-mediated transpeptidation bonding. In another example, a template polynucleotide may be modified during sample preparation to include one of a Snap-tag sequence or O⁶-Benzylguanine, and a single template site of a scaffold may include the other of the two, to permit covalent bonding between the two in accordance with the Snap-tag/O⁶-Benzylguanine system. In another example, a template polynucleotide may be modified during sample preparation to include one of a CLIP-tag sequence or O²-benzylcytosine, and a single template site of a scaffold may include the other of the two, to permit covalent bonding between the two in accordance with the CLIP-tag/O²-benzylcytosine system, and the CLIP-tag/O²-benzylcytosine system.

Any of the foregoing examples may likewise be used for attachment of one or more accessories to one or more accessory sites on a scaffold. For attachment to an accessory site of a DNA scaffold or of a polypeptide scaffold, an accessory (such as an accessory oligo-DNA) may have a complementary attachment moiety or structure added thereto. In an example, a nucleotide sequence may be included in or attached to an accessory and may include a complementary attachment moiety or structure being attached thereto or included therein.

For example, for a dendrimer DNA scaffold, a nucleotide sequence may be included in or attached to an accessory and the sequence may be complementary to an adapter of a dendrimer DNA scaffold, such as to downstream overhangs of the adapter of the last generation of the dendrimer DNA scaffold, or to otherwise unhybridized downstream overhangs of adapters of other generations of the dendrimer.

In another example, an accessory (such as an accessory oligo-DNA) may include or be attached to a nucleotide sequence, such as extending from one of its ends in the case of an accessory oligo-DNA, and the sequence is complementary to another sequence which other sequence is included in or attached to accessory sites of the scaffold. Watson-Crick base-pairing between the complementary sequences results in hybridization and bonding between the two and, thus, attachment of accessories to accessory bonding sites. In another example, the accessory may include covalent modification thereof to permit covalent attachment to it of a moiety or structure that is complementary thereto. For example, modifications to a nucleotide included in a template polynucleotide, such as on a phosphate group, the base, or the sugar, may be included to provide a site for covalent attachment to an accessory site of a scaffold. Accessory sites of the scaffold may in turn include a complementary moiety or structure permitting attachment to accessories such as oligo-DNA accessories. In an example, nucleotides modified to include an attachment moiety with which a complementary moiety of an accessory bonding site of a scaffold may be included in a polynucleotide sequence added to or included in an accessory such as an accessory oligo-DNA to permit bonding between them. Numerous modified nucleotides bearing such chemical moieties are commercially available for covalent attachment of compositions to DNA molecules in which such modified nucleotides have been incorporated.

In another example, an accessory may be modified by attaching to it a polypeptide. Such polypeptide may possess an amino acid sequence and/or structure so as to be complementary to an amino acid structure of an accessory site of a scaffold, such that the accessories may attach, via their attached polypeptides, to the accessory sites of the scaffold. Examples of pairs of polypeptides for covalent or noncovalent bonding between accessory sites and accessories were provided above and include, as non-limiting examples, alpha-helical amino acid sequences with heptad repeats for formation of coiled coil attachments to one another, biotin-avidin binding pairs, SpyTag/SpyCatcher system, LPXTG/oligoglycine nucleophilic pairs for sortase-mediated transpeptidation bonding. In another example, an accessory, such as an accessory oligo-DNA, may be modified to include one of a Snap-tag sequence or O⁶-Benzylguanine, and accessory sites of a scaffold may include the other of the two, to permit covalent bonding between the two in accordance with the Snap-tag/O⁶-Benzylguanine system. In another example, an accessory may be include one of a CLIP-tag sequence or O²-benzylcytosine, and accessory sites of a scaffold may include the other of the two, to permit covalent bonding between the two in accordance with the CLIP-tag/O²-benzylcytosine system.

Attachment of a template polynucleotide to a template site of a scaffold, or of an accessory such as an accessory oligo-DNA to an accessory site of a scaffold, may be through direct bonding therebetween. In other examples, spacers, polymers, or other chemical compositions may be included connecting a nucleotide of a DNA scaffold, or an amino acid of a polypeptide scaffold, to a single template site or accessory site or both. In an example, a moiety or structure for bonding between a template polynucleotide or accessory may be incorporated into a modified amino acid of a polypeptide scaffold or a modified nucleotide of a DNA scaffold, and may bond directly to a complementary moiety or structure attached directly to a template polynucleotide or accessory. In another example, a spacer, polymer, or other chemical composition may extend from a nucleotide of a DNA scaffold or an amino acid of a polypeptide scaffold, or both, and a moiety or structure for bonding a template polynucleotide or accessory may be present on the spacer, polymer, or other chemical moiety at a distance from the attachment of said spacer, polymer, or other chemical moiety to the scaffold. In another example, a spacer, polymer, or other chemical composition may extend from template polynucleotide, or an accessory, or both, and a moiety or structure for bonding a scaffold may be present on the spacer, polymer, or other chemical moiety at a distance from the attachment of said spacer, polymer, or other chemical moiety to the template polynucleotide or accessory. In an example, such a spacer, polymer, or other chemical composition may extend from a scaffold to a single template site and from a template polynucleotide, or from a scaffold to an accessory site and from an accessory, or from a scaffold to a single template site and from a template polynucleotide and from a scaffold to an accessory site and from an accessory. In another example, such a spacer, polymer, or other chemical composition may extend from any one of or any combination of two or more of a scaffold to a single template site, a template polynucleotide, a scaffold to an accessory site, and an accessory.

A spacer, polymer, or chemical compositions that may extend from any one of or any combination of two or more of a spacer to a single template site, a template polynucleotide, a spacer to an accessory site, or an accessory, and no two such spacers, polymers, or chemical compositions must be the same spacers, polymers, or chemical compositions as each other, although they may. In an example, a spacer, polymer, or other chemical composition may extend from a DNA scaffold or polypeptide scaffold and the spacer, polymer, or other chemical composition may include more than one accessory site.

In some examples, polymers by which an accessory, such as an accessory oligo-DNA, are attached to a scaffold including an accessory site, may be random, block, linear, and/or branched copolymers comprising two or more recurring monomer units in any order or configuration, and may be linear, cross-linked, or branched, or a combination thereof. In an example, the polymer may be a heteropolymer and the heteropolymer may include an acrylamide monomer, such as

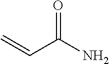

or a substituted analog thereof (“substituted” referring to the replacement of one or more hydrogen atoms in a specified group with another atom or group). In an example, the polymer is a heteropolymer and may further include an azido-containing acrylamide monomer. In some aspects, the heteropolymer includes:

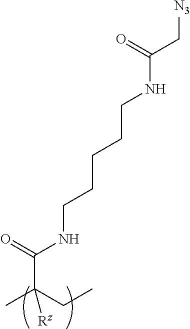

and optionally

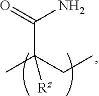

where each R^{z} is independently H or C₁₋₄ alkyl.

In an example, a polymer used may include examples such as a poly(N-(5-azidoacetamidylpentyl)acrylamide-co-acrylamide), also known as PAZAM:

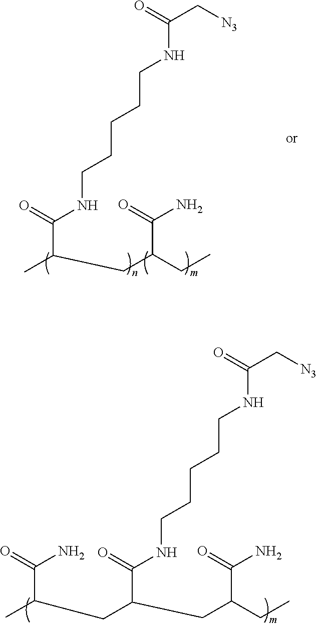

wherein n is an integer in the range of 1-20,000, and m is an integer in the range of 1-100,000

In some examples, the acrylamide monomer may include an azido acetamido pentyl acrylamide monomer:

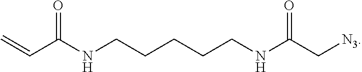 [SMILES: C=CC(=O)NCCCCCNC(=O)CN=[N+]=[N-]]

In some examples, the acrylamide monomer may include an N-isopropylacrylamide

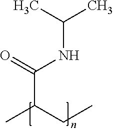

In some aspects, the heteropolymer may include the structure:

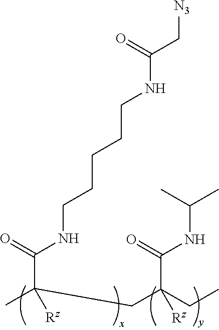

wherein x is an integer in the range of 1-20,000, and y is an integer in the range of 1-100,000, or

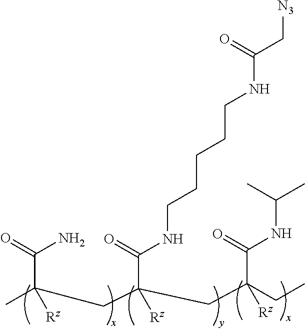

wherein y is an integer in the range of 1-20,000 and x and z are integers wherein the sum of x and z may be within a range of from 1 to 100,000, where each R^{z} is independently H or C₁₋₄ alkyl and a ratio of x:y may be from approximately 10:90 to approximately 1:99, or may be approximately 5:95, or a ratio of (x:y):z may be from approximately 85:15 to approximately 95:5, or may be approximately 90:10 (wherein a ratio of x:(y:z) may be from approximately 1:(99) to approximately 10:(90), or may be approximately 5:(95)), respectively. In these examples, approximately means relative amounts of one may differ from amounts stated in the listed rations by up to 5%.

A “heteropolymer” is a large molecule of at least two different repeating subunits (monomers). An “acrylamide monomer” is a monomer with the structure

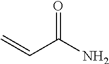

or a substituted analog thereof (e.g., methacrylamide or N-isopropylacrylamide). An example of a monomer including an acrylamide group and the azido group is azido acetamido pentyl acrylamide shown above. “Alkyl” refers to a straight or branched hydrocarbon chain that is fully saturated (i.e., contains no double or triple bonds). Example alkyl groups include methyl, ethyl, propyl, isopropyl, butyl, isobutyl, and tertiary butyl. As an example, the designation “C₁₋₄ alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

One or more of any one or more of the foregoing polymers may be attached to a DNA scaffold or polypeptide scaffold as one or more accessories or for attaching one or more accessories to the scaffold, such as accessory oligo-DNA molecules. For example, a scaffold may contain one or more alkyne groups, or one or more other groups that may react and bond with an azide group such as a norbornene group, and an azide of a polymer may bond covalently with the alkyne, norbornene, or other group or groups of the scaffold via cycloaddition click chemistry reaction. In a further example, other compositions or additional accessories such as other compositions or structures, including as an example oligo-DNA molecules, may also contain or be modified to contain one or more alkyne groups, or one or more other groups that may react and bond with an azide group such as a norbornene group, and an azide of a polymer may bond covalently with the alkyne, norbornene, or other group or groups of such other compound or additional accessories via cycloaddition click chemistry reaction. One or more polymers may thus be attached to a scaffold, and one or more such attached polymer may further attach to one or more further compositions such as additional accessories, such as oligo-DNA molecules. In other examples, reactive chemistries may be used for attaching a polymer to a scaffold and accessories such as oligo-DNA molecules to a polymer.

In an example, a single template polynucleotide may be bound to a single template site of a scaffold, and multiple accessory nucleotides, such as accessory oligo-DNA molecules, may be bound to accessory sites of a scaffold (whether directly, or via a polymer as disclosed above, or other polymer, or spacer or other composition). Examples of such oligo-DNA molecules may be primers for performing clustering on the scaffold. As part of a conventional clustering process, copies of a template polynucleotide or its complement are made on a surface of a substrate. As explained above, in some instances such on-surface clustering may unfavorably result in formation of one or more polyclonal clusters. As disclosed herein, clustering may be performed on a scaffold, such as in solution, without prior attachment of the scaffold to a surface. In other examples, a scaffold with a single template polynucleotide attached may be attached to a surface of a substrate and clustering may then be performed on the surface of the substrate, on the scaffold, or on the scaffold and on the surface of the substrate.

For a clustering procedure, a modification may be made to a template polynucleotide such as during sample preparation to include one or more nucleotide sequences at one or both of its 3-prime and 5-prime ends. A copy or copies of the template nucleotide and nucleotide sequences complementary to the template nucleotide may then be synthesized on, as disclosed herein, a scaffold, forming a cluster. Such on-scaffold clustering may result in formation of a monoclonal cluster.

For example, a template polynucleotide may bond to a single template attachment site with its 5-prime end oriented towards the scaffold and its 3-prime end oriented away from the site of bonding to the scaffold. The 3-prime end may include a nucleotide sequence that is complementary to a nucleotide sequence included in a first primer. A “primer” is defined as a single stranded nucleic acid sequence (e.g., single strand DNA or single strand RNA) that serves as a starting point for DNA or RNA synthesis. A primer can be any number of bases long and can include a variety of non-natural nucleotides. In an example, the primer is a short strand, ranging from 20 to 40 bases, or 10 to 20 bases. Copies of primers complementary to the 3-prime end of the template polynucleotide may further be attached to accessory sites of the scaffold, directly or by attachment to a polymer (such as PAZAM or related polymers disclosed herein, as non-limiting examples), spacer, or other chemical composition as disclosed herein.

A polymerization reaction may then be performed, in which the 3-prime end of the template polynucleotide hybridizes via Watson-Crick base pairing to a scaffold-bound first primer complementary thereto. A polymerase in the polymerization reaction may create a nascent strand complement to the template polynucleotide as attached to the scaffold, initiated from the scaffold-attached primer to which the 3-prime end of the template polynucleotide is hybridized. The template polynucleotide and its complement may then be dehybridized.

The complement to the template polynucleotide, at the 3-prime end of the complement, may include a nucleotide sequence that is complementary to a second primer sequence. Copies of second primers complementary to the 3-prime end of the complement to the template polynucleotide may further be attached to accessory sites of the scaffold. A second polymerization reaction may then be performed, in which the 3-prime end of the template polynucleotide hybridizes via Watson-Crick base pairing to a scaffold-bound first primer complementary thereto and the 3-prime end of the complement to the template polynucleotide hybridizes via Watson-Crick base pairing to a scaffold-bound second primer complementary thereto. A polymerase in the second polymerization reaction may create another nascent strand complement to the template polynucleotide as attached to the scaffold, initiated from the scaffold-attached first primer to which the 3-prime end of the template polynucleotide is hybridized. And the polymerase in the second polymerization reaction may further create a nascent strand copy of the template polynucleotide as attached to the scaffold, initiated from the scaffold-attached second primer to which the 3-prime end of the complement to the template polymerized in the prior polymerization reaction is hybridized. The template polynucleotide and copy thereof and its complements may then be dehybridized.

Subsequent polymerization reactions may then be performed in an iterative process. 3-prime ends of scaffold-bound template polynucleotide and copies thereof hybridize to scaffold-bound first primers complementary thereto, and 3-prime ends of scaffold-bound complements to the template polynucleotide hybridize to scaffold-bound second primers complementary thereto. Nascent strands are polymerized by a polymerase, initiated at the scaffold-bound first and second primers to which the scaffold bound template polynucleotide and complements to and copies thereof are hybridized. Following dehybridization of the strands following polymerization, successive polymerization reactions are performed, thereby multiplying the number of copies of template polynucleotide and complements thereto attached to the scaffold. In this manner, copies of and complements to the template polynucleotide are amplified, with the amplified copies bound to the scaffold, forming a cluster. As disclosed herein, this clustering process may be performed on a scaffold, such as in solution, as opposed to conventional clustering which is performed on a surface of a substrate in a conventional SBS process. Because there are copies of and complements to only a single template polynucleotide clustered on the scaffold, a monoclonal cluster is present on the scaffold.

In such an example, where a sequence at or attached to the 5-prime end of a template polynucleotide bonds to a single template site, orienting the 3-prime end of the template polynucleotide away from the scaffold, the template polynucleotide may bond to the single template site of the scaffold by hybridization to a primer sequence attached to or part of the single template site, referred to as a template site primer. In an example, a template polynucleotide, as prepared by a sample preparation process, may have at or attached to its 5-prime end a nucleotide sequence complementary to the template site primer. 3-prime to such nucleotide sequence complementary to the template site primer, the template polynucleotide may include a nucleotide sequence that corresponds to the nucleotide sequence of the above-described second primer (the second primer being a scaffold-attached primer to which a 3-prime end of a complement to the template polynucleotide may hybridize by complementary Watson-Crick base pairing). Inclusion of such sequence in the template polynucleotide means that a complement to the template polynucleotide, synthesized during a polymerization step, would have, towards its 3-prime end, a polynucleotide sequence that is complementary to the sequence of such second primer. Having such sequence towards the 3-prime end of a complement to a template polynucleotide enables hybridization of the 3-prime end of the complement to such second primer during a subsequent polymerization reaction during clustering.

At the 3-prime end of the template polynucleotide, oriented away from the template polynucleotide's 5-prime end bound to the single template site, the template polynucleotide may include a sequence complementary to the first primer as described above. During a first polymerization step, as described above, such nucleotide sequence at the template polynucleotide's 3-prime end may hybridize to a first primer, followed by polymerization of a nascent complement to the template polynucleotide. It may be advantageous for there to be a discontinuation of polymerization of a complement to the template polynucleotide between the portion of the template polynucleotide hybridized to the template site primer and a nucleotide sequence located 3-prime thereto in the template polynucleotide that includes the sequence of the second primer. That is, it may be advantageous for the complement of the template polynucleotide to have at its 3-prime end a sequence complementary to the second primer. However, if there is no discontinuation of polymerization after adding to the nascent complement to the template polynucleotide a nucleotide sequence complementary to the sequence corresponding to the second primer, the 3-prime end of the complement to the template polynucleotide would not end there.

For example, if a nucleotide sequence complementary to the template site primer is 5-prime to and contiguous with the sequence complementary to the second primer, the 3-prime end of the complement to the template polynucleotide may include a nucleotide sequence included in the template site primer. For example, a DNA polymerase, in polymerizing the complement to the template polynucleotide, may displace the template site primer from hybridization to the 5-prime end of the template polynucleotide and polymerize the addition of a nucleotide sequence corresponding thereto to the 3-prime end of the complement to the template polynucleotide. Such an outcome may be unwanted if it impaired an ability of the 3-prime end of the complement to the template polynucleotide from hybridizing to an above-described second primer at an accessory site.

In an example it may therefore be desirable to incorporate a discontinuation of polymerization 3-prime to the 5-prime end of the template polynucleotide where such 5-prime end of the template polynucleotide bonds to the single template site by hybridizing to a template site primer. For example, a linker, such as a PEG linker, alkyl linker, or other chemical moiety may be included to connect the nucleotide sequence that hybridizes to the template site primer to the 5-prime end of the template polynucleotide. The presence of such a linker, rather than a contiguous nucleotide sequence connection, would prevent a polymerase from adding a nucleotide sequence corresponding to the template site primer to the 3-prime end of the complement of the template polynucleotide, which would instead end with a nucleotide sequence complementary to the nucleotide sequence of the second primer as may be desired.

In other examples, a template polynucleotide may have or be attached to a polynucleotide sequence at the template polynucleotide's 3-prime end that is complementary to a primer that is part of or attached to a single template site of a scaffold, referred to as a template site primer. Following hybridization of such sequence of or attached to the 3-prime end of the template polynucleotide to template site primer, a polymerization process may be performed wherein a DNA polymerase polymerizes formation of a nascent polynucleotide complementary to the template polynucleotide, initiated from the template site primer. Dehybridization of the template polynucleotide from the scaffold-attached complement to the template polynucleotide is then performed. The 3-prime end of the scaffold-attached complement to the template polynucleotide, oriented away from the site of attachment to the scaffold, may include a nucleotide sequence that is complementary to the above-described second primer sequence (the second primer being a scaffold-attached primer to which a 3-prime end of a complement to the template polynucleotide may hybridize by complementary Watson-Crick base pairing). Copies of second primers complementary to the 3-prime end of the complement to the template polynucleotide may further be attached to accessory sites of the scaffold. A second polymerization reaction may then be performed, in which the 3-prime end of the complement to the template polynucleotide hybridizes via Watson-Crick base pairing to a scaffold-bound second primer complementary thereto. A polymerase in the second polymerization reaction may create a nascent strand copy of the template polynucleotide (i.e., a complement to the scaffold-bound complement to the template polynucleotide), initiated from the scaffold-attached second primer to which the 3-prime end of the complement to the template polymerized in the prior polymerization reaction is hybridized. A dehybridization step may then be performed to dehybridize the scaffold bound complement to the template polynucleotide and copy of the template polynucleotide from each other.

The copy of the template polynucleotide, at the 3-prime end of the copy, may include a nucleotide sequence that is complementary to the above-described first primer sequence. Copies of first primers complementary to the 3-prime end of the copy of the template polynucleotide, described above, may further be attached to accessory sites of the scaffold. A third polymerization reaction may then be performed, in which the 3-prime end of the copy of the template polynucleotide hybridizes via Watson-Crick base pairing to a scaffold-bound first primer complementary thereto and the 3-prime end of the complement to the template polynucleotide hybridizes via Watson-Crick base pairing to a scaffold-bound second primer complementary thereto. A polymerase in the third polymerization reaction may create another nascent strand complement to the template polynucleotide as attached to the scaffold, initiated from the scaffold-attached first primer to which the 3-prime end of the copy of the template polynucleotide is hybridized. And the polymerase in the third polymerization reaction may further create a nascent strand copy of the template polynucleotide, initiated from the scaffold-attached second primer to which the 3-prime end of the complement to the template polymerized in the prior polymerization reaction is hybridized. A dehybridization step dehybridizing the copies of and complements to the template polynucleotide from each other may then be performed.

Subsequent polymerization reactions may then be performed in an iterative process. 3-prime ends of scaffold-bound copies of template polynucleotide hybridize to scaffold-bound first primers complementary thereto, and 3-prime ends of scaffold-bound complements to the template polynucleotide hybridize to scaffold-bound second primers complementary thereto. Nascent strands are polymerized by a polymerase, initiated at the scaffold-bound first and second primers to which the scaffold bound template polynucleotide and complements to and copies thereof are hybridized. Dehybridization of the strands is performed following polymerization, then successive polymerization reactions are performed followed by further dehybridization. In this manner, copies of and complements to the template polynucleotide are amplified, with the amplified copies and complements bound to the scaffold, forming a cluster. As disclosed herein, this clustering process may be performed on a scaffold, such as in solution, as opposed to conventional clustering which is performed on a surface of a substrate in a conventional SBS process. Because there are copies of and complements to only a single template polynucleotide clustered on the scaffold, a monoclonal cluster is present on the scaffold.

In an example, an end of a template polynucleotide includes or is attached to a nucleotide sequence that is complementary to a nucleotide sequence included in or attached to the single template site of the scaffold, referred to as the third template-site primer. In an example, a complement to the template polynucleotide may be synthesized on the scaffold initiated at the third template site primer.

In examples of on-scaffold clustering as disclosed herein, a scaffold may be a DNA scaffold or a polypeptide scaffold as disclosed herein. A template polynucleotide may be bound to a single template site of a scaffold according to any of various covalent or non-covalent bonds disclosed herein. For example, either end of a template polynucleotide may include a moiety or structure from a bonding site pair such as identified in Table 1, and the complementary moiety or structure of the same pair may be present at the single template site of the scaffold. Successive rounds of polymerization may then follow much as described above. For example, a 3-prime end of a template polynucleotide bound to the scaffold's single template site at or towards the template polynucleotide's 5-prime end may hybridize to an oligonucleotide primer bound to an accessory site of scaffold and a complement thereto synthesized by a DNA polymerase. Successive rounds of polymerization may then follow as described above, resulting in polymerization of multiple copies of the template polynucleotide and complements thereto emanating from accessory sites of the scaffold. Because only a single template polynucleotide was bound to the scaffold, the scaffold having only a single template polynucleotide site, such copies would constitute a monoclonal cluster on the scaffold.

In another example, a scaffold may attach to a surface of a substrate, such as a surface of a substrate for use in an SBS procedure. For example, accessory sites of a scaffold may include or be or become attached to sites attached to a surface of a substrate, or compositions that bond to a surface of a substrate. In an example, a surface of a substrate may be bound to primers, such as for example copies of primers that are complementary to first primers or second primers as described above, or both, as non-limiting examples. Such complementary primers may be attached either directly to a surface of a substrate or may be attached to a modified surface, such as a surface to which polymer molecules have been attached (e.g., PAZAM or related polymers) with primers attached to such polymers. Aforementioned first primers and second primers may be attached to accessory sites of a scaffold (directly, or via a polymer such as PAZAM or other PAZAM-like polymers as disclosed above as non-limiting examples, or spacer or other composition). Such first and second primers of or attached to a scaffold may hybridize to primers complementary thereto as attached to a surface of a substrate, thereby bonding a scaffold to the surface of the substrate.

Examples of first and second primers as discussed above may include primers used in existing SBS processes. Specific examples of suitable primers include P5 and/or P7 primers, which are used on the surface of commercial flow cells sold by Illumina, Inc., for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms. And portion of a template polynucleotide that includes a nucleotide sequence corresponding to, or complementary to, a first or second primer as disclosed above may have, for example, a sequence corresponding to or complementary to a P5 primer (including a nucleotide sequence of AATGATACGGCGACCACCGAGATCTACAC (SEQ ID NO:7)), a P7 primer (including a nucleotide sequence of CAAGCAGAAGACGGCATACGAGAT (SEQ ID NO:8)), or both, in accordance with such primer sequences as used in the above-mentioned SBS platforms, or others.

A substrate for an SBS process may include, as non-limiting examples, substrates used in any of the aforementioned SBS platforms or others. As a non-limiting example, such a substrate may be a flow cell. As used herein, the term “flow cell” is intended to mean a vessel having a chamber (i.e., flow channel) where a reaction can be carried out, an inlet for delivering reagent(s) to the chamber, and an outlet for removing reagent(s) from the chamber. In some examples, the chamber enables the detection of a reaction or signal that occurs in the chamber. For example, the chamber can include one or more transparent surfaces allowing for the optical detection of arrays, optically labeled molecules, or the like, in the chamber. As used herein, a “flow channel” or “flow channel region” may be an area defined between two bonded components, which can selectively receive a liquid sample. In some examples, the flow channel may be defined between a patterned support and a lid, and thus may be in fluid communication with one or more depressions defined in the patterned support. In other examples, the flow channel may be defined between a non-patterned support and a lid.

As used herein, the term “depression” refers to a discrete concave feature in a patterned support having a surface opening that is completely surrounded by interstitial region(s) of the patterned support surface. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As an example, the depression can be a well. Also as used herein, a “functionalized depression” refers to the discrete concave feature where primers are attached, in some examples being attached to the surface of the depression by a polymer (such as a PAZAM or similar polymer).

The term flow cell “support” or “substrate” refers to a support or substrate upon which surface chemistry may be added. The term “patterned substrate” refers to a support in which or on which depressions are defined. The term “non-patterned substrate” refers to a substantially planar support. The substrate may also be referred to herein as a “support,” “patterned support,” or “non-patterned support.” The support may be a wafer, a panel, a rectangular sheet, a die, or any other suitable configuration. The support is generally rigid and is insoluble in an aqueous liquid. The support may be inert to a chemistry that is used to modify the depressions. For example, a support can be inert to chemistry used to form a polymer coating layer, to attach primers such as to a polymer coating layer that has been deposited, etc. Examples of suitable supports include epoxy siloxane, glass and modified or functionalized glass, polyhedral oligomeric silsequioxanes (POSS) and derivatives thereof, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon, ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (TaO₅) or other tantalum oxide(s) (TaOₓ), hafnium oxide (HaO₂), carbon, metals, inorganic glasses, or the like. The support may also be glass or silicon or a silicon-based polymer such as a POSS material, optionally with a coating layer of tantalum oxide or another ceramic oxide at the surface. A POSS material may be that disclosed in Kejagoas et al., Microelectronic Engineering 86 (2009) 776-668, which is incorporated by reference herein in its entirety.

In an example, depressions may be wells such that the patterned substrate includes an array of wells in a surface thereof. The wells may be micro wells or nanowells. The size of each well may be characterized by its volume, well opening area, depth, and/or diameter.

Each well can have any volume that is capable of confining a liquid. The minimum or maximum volume can be selected, for example, to accommodate the throughput (e.g., multiplexity), resolution, analyte composition, or analyte reactivity expected for downstream uses of the flow cell. For example, the volume can be at least about 1×10⁻³ μm³, about 1×10⁻² μm³, about 0.1 μm³, about 1 μm³, about 10 μm³, about 100 μm³, or more. Alternatively or additionally, the volume can be at most about 1×10⁴ μm³, about 1×10³ μm³, about 100 μm³, about 10 μm³, about 1 μm³, about 0.1 μm³, or less.

The area occupied by each well opening on a surface can be selected based upon similar criteria as those set forth above for well volume. For example, the area for each well opening on a surface can be at least about 1×10⁻³ μm², about 1×10⁻² μm², about 0.1 μm², about 1 μm², about 10 μm², about 100 μm², or more. Alternatively or additionally, the area can be at most about 1×10³ μm², about 100 μm², about 10 μm², about 1 μm², about 0.1 μm², about 1×10⁻² μm², or less. The area occupied by each well opening can be greater than, less than or between the values specified above.

The depth of each well can be at least about 0.1 μm, about 1 μm, about 10 μm, about 100 μm, or more. Alternatively or additionally, the depth can be at most about 1×10³ μm, about 100 μm, about 10 μm, about 1 μm, about 0.1 μm, or less. The depth of each well 14′ can be greater than, less than or between the values specified above.

In some instances, the diameter of each well can be at least about 50 nm, about 0.1 μm, about 0.5 μm, about 1 μm, about 10 μm, about 100 μm, or more. Alternatively or additionally, the diameter can be at most about 1×10³ μm, about 100 μm, about 10 μm, about 1 μm, about 0.5 μm, about 0.1 μm, or less (e.g., about 50 nm). The diameter can be about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 900 nm, about 950 nm, about 1 μm, about 1.25 μm, about 1.5 μm, about 1.74 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.25 μm, about 5.5 μm, about 5.75 μm, about 6 μm, about 6.25 μm, about 6.5 μm, about 6.75 μm, about 7 μm, about 7.25 μm, about 7.5 μm, about 7.75 μm, about 8 μm, about 8.25 μm, about 8.5 μm, about 8.75 μm, about 9 μm, about 9.25 μm, about 9.5 μm, or about 9.75 μm. The diameter of each well can be greater than, less than or between the values specified above. A nanowell as the term is used herein is intended to mean a well with a round opening whose largest diameter is about 1 μm or less.

It is to be understood that the ranges provided herein include the stated range and any value or sub-range within the stated range. For example, a range from about 100 nm to about 1 μm (1000 nm), should be interpreted to include not only the explicitly recited limits of from about 100 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

In an example, a size of a nanoparticle may be such that presence of the nanoparticle in a well such as a nanowell occupies so much of the well's volume that another nanoparticle cannot occupy the well at the same time. Size of a nanoparticle may be designed or determined, in reference to a known size of wells in a surface of a substrate, such that it may enter a well in which no other nanoparticle is present but whose entry into a well would be prevented by presence of another nanoparticle that previously entered and still is present in the well. Nanoparticles sized so as not to be able to fit more than two to a well may promote monoclonality of a cluster within a well. For example, in a conventional SBS process, template polynucleotides may be introduced to a flow cell patterned with wells in a solution in a concentration calibrated to maximize the number of wells in which a template polynucleotide will seed (i.e., bind, such as to a primer attached to the well, directly or via a surface-attached polymer, that is complementary to an nucleotide sequence of part of a template nucleotide), but low enough as to minimize as much as possible the formation of polyclonal clusters.

In an example, a flow cell may include nano-scale regions that are not depressions or nanowells but otherwise spatially isolated regions within which a template polynucleotide or scaffold may bind, or seed, referred to herein as nanopads. In some examples, a flow cell surface includes nanopads, separated from each other by regions of surface where a template polynucleotide or scaffold may not bind. Nanopads may be spaced from one another so as to promote formation of monoclonal clusters. For example, nanopads may be separated from each other such that a cluster formed within one nanopad seeded by a single template polynucleotide would be separated sufficiently from another such nanopad that was seeded by only one template polynucleotide. However, it may be difficult to prevent the seeding of a nanopad by more than one template polynucleotide, resulting in one or more polyclonal clusters forming. In an example as disclosed herein, a nanoparticle may promote formation of monoclonal clusters in favor of polyclonal clusters by preventing more than one template polynucleotide from seeding or attaching within a given nanopad. For example, a size of a nanoparticle may be such that there is insufficient room on a nanopad for more than one nanoparticle to bind, where template polynucleotides bond to a single template polynucleotide sites of scaffolds.

In some instances, a polyclonal cluster may occur if two or more template polynucleotides with nucleotide sequences that differ from each other bind within, or seed, the same well as each other. Molecules may distribute among wells based on their concentration within an applied solution on the basis of a Poisson distribution, according to which there is a balance between minimizing the number of unoccupied wells (for increased efficiency of an SBS run) while minimizing a number of wells occupied by multiple, disparate template polynucleotides. Disparity between a minimum well size and a size of a template polynucleotide (e.g., a diameter of a B-DNA molecule may be on the order of 2 nm) may result in choosing between a concentration that does not utilize as much substrate surface, such as surface within wells, as available or preferred on the one hand and resulting in formation of an undesirable or undesirably high number of polyclonal clusters.

As disclosed herein, template polynucleotides may bond to a nanoparticle, with only one template polynucleotide bonding per nanoparticle. A nanoparticle may be sized so as to permit entry of a nanoparticle in a well of a flow cell in which another nanoparticle is non already present, but not to enter a well of a flow cell in which another nanoparticle is already present. Clustering, such as monoclonal clustering, may occur on a nanoparticle before a nanoparticle enters a well, resulting in monoclonal clusters being present in wells. Or, a template polynucleotide may bond to a template site of a nanoparticle and the nanoparticle may enter and bind within a well (for example, by binding of accessory sites to the surface or modification to the surface of a well), thereby seeding the well with only a single nanoparticle, and clustering may then proceed within the well, resulting in monoclonal clusters being present in wells. In some examples, some degree of clustering may occur on nanoparticles before they enter a well and further clustering may occur after the nanoparticle enters a well. All such examples include examples where monoclonal clusters form within wells. Furthermore, tuning a size of nanoparticles so as to reduce, minimize, or in an example eliminate the simultaneous presence of more than one nanoparticle in a well at one time may reduce, minimize, or in an example eliminate formation of polyclonal clusters.

Nanoparticle size may be tuned by modifying a size of a scaffold, modifying a size of accessories bonded to accessory sites such as polymers attached thereto, or both. Size of a nanoparticle may also be modified by an amount of clustering that has or has not occurred on the nanoparticle, such as by modifying a number of sites on a nanoparticle upon which copies of and complements to a template polynucleotide may bind during rounds of polymerization during clustering, with fewer such sites potentially resulting in a lower upper limit of nanoparticle size and more such sites potentially resulting in a larger upper limit of nanoparticle size. A number of rounds of polymerization during clustering may also modify nanoparticle size, with more rounds resulting in more copies of and complements to a template polynucleotide bound to the nanoparticle and therefore potentially increasing its upper size limit and fewer rounds resulting in fewer copies of and complements to a template polynucleotide bound to a nanoparticle and thus potentially reducing its upper size limit. A size of a nanoparticle may be determine according to its size before clustering on a scaffold has occurred or after clustering on a scaffold has occurred.

As used herein the term “nanoparticle” is intended to mean a particle with a largest dimension up to about 1,000 nm in size. Depending on the geometry, the dimension may refer to the length, width, height, diameter, etc. Although “diameter” is generally used to describe the dimension as one example herein, the nanoparticle described herein need not be spherical or circular. A nanoparticle as disclosed herein may have a diameter of about 2 nm, about 5 nm, about 7 nm, about 10 nm, about 12 nm, about 15 nm, about 17 nm, about 20 nm, about 22 nm, about 25 nm, about 27 nm, about 30 nm, about 32 nm, about 35 nm, about 40 nm, about 42 nm, about 45 nm, about 47 nm, about 50 nm, about 52 nm, about 55 nm, about 57 nm, about 60 nm, about 62 nm, about 65 nm, about 67 nm, about 70 nm, about 72 nm, about 75 nm, about 77 nm, about 80 nm, about 82 nm, about 85 nm, about 87 nm, about 90 nm, about 92 nm, about 95 nm, about 97 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1,000 nm. Diameter of a nanoparticle is measured by dynamic light scattering (DLS), also known as quasi-elastic light scattering, expressed as twice the hydrodynamic radius (Rh), which may be determined on a DLS system or other system that includes DLS and other functionality (e.g., a ZETASIZER®, Malvern Instruments Limited).

A nanoparticle as disclosed herein may have a diameter within a range of about 2 nm to about 10 nm, about 5 nm to about 15 nm, about 7 nm to about 20 nm, about 10 nm to about 25 nm, about 15 nm to about 30 nm, about 20 nm to about 50 nm, about 40 nm to about 60 nm, about 50 nm to about 75 nm, about 60 nm to about 100 nm, about 70 nm to about 100 nm, about 75 nm to about 100 nm, about 80 nm to about 110 nm, about 90 nm to about 130 nm, about 100 nm to about 150 nm, about 100 nm to about 200 nm, about 150 nm to about 225 nm, about 200 nm to about 250 nm, about 200 nm to about 300 nm, about 225 nm to about 275 nm, about 250 nm to about 300 nm, about 275 nm to about 325 nm, about 300 nm to about 400 nm, about 300 nm to about 350 nm, about 325 nm to about 375 nm, about 350 nm to about 400 nm, about 375 nm to about 425 nm, about 400 nm to about 500 nm, about 400 nm to about 450 nm, about 425 nm to about 475 nm, about 450 nm to about 500 nm, about 475 nm to about 525 nm, about 500 nm to about 600 nm, about 500 nm to about 550 nm, about 525 nm to about 575 nm, about 550 nm to about 600 nm, about 575 nm to about 625 nm, about 600 nm to about 700 nm, about 600 nm to about 625 nm, about 625 nm to about 675 nm, about 650 nm to about 700 nm, about 675 nm to about 725 nm, about 700 nm to about 800 nm, about 700 nm to about 725 nm, about 725 nm to about 775 nm, about 750 nm to about 800 nm, about 775 nm to about 825 nm, about 800 nm to about 900 nm, about 800 nm to about 850 nm, about 825 nm to about 875 nm, about 850 nm to about 900 nm, about 875 nm to about 925 nm, about 900 nm to about 1,000 nm, about 900 nm to about 950 nm, about 925 nm to about 975 nm, about 950 nm to about 1,000 nm, about 300 nm to about 450 nm, about 350 nm to about 500 nm, about 400 nm to about 550 nm, about 450 nm to about 600 nm, about 500 nm to about 650 nm, about 550 nm to about 700 nm, about 600 nm to about 750 nm, about 650 nm to about 800 nm, about 700 nm to about 850 nm, about 750 nm to about 900 nm, about 800 nm to about 950, or about 850 nm to about 1,000 nm.

### Non-Limiting Examples

The following examples are intended to illustrate particular examples of the present disclosure, but are by no means intended to limit the scope thereof.

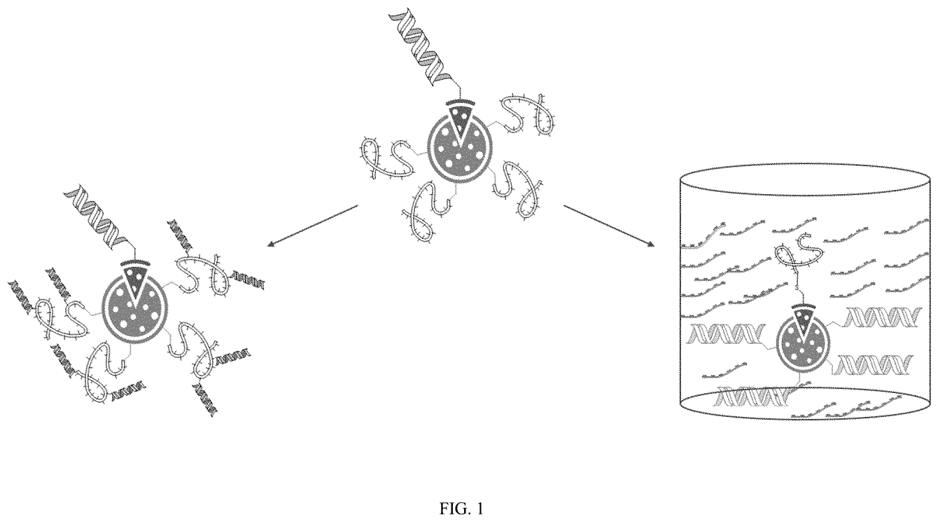

FIG. 1 shows an illustration of a non-limiting example of a nanoparticle as disclosed herein. In this non-limiting example, a single template polynucleotide site is shown as a wedge-shaped portion of a scaffold portion of a nanoparticle as disclosed. A single template polynucleotide is shown bound to the single template site. Also in this non-limiting example, a plurality of accessories are shown extending from accessory sites of the scaffold. In the center illustration, the accessories are shown as polymers. In the left panel, a number of copies of polynucleotides complementary to the template polynucleotide and copes of the template polynucleotide are shown, with such copies attached to and extending from the scaffold. In this example, they extend from the polymers, which in turn extend from the scaffold.

In the right panel, a nanoparticle with a template polynucleotide bound thereto at the single template site is shown in a well of a substrate. A plurality of accessory oligonucleotides are shown extending from the scaffold. Although not shown in the right-hand panel, in this example the accessory oligonucleotides extend from the polymers that are attached to the scaffold. In other examples, the accessory oligonucleotides may extend directly from a scaffold without an intervening polymer being present therebetween. Nucleotide sequences of the accessory oligonucleotides are complementary to primers attached to the surface of the well. The accessory oligonucleotides thereby hybridize to the well-attached primers and attach to the surface of the well. Here, only one nanoparticle can be present in the well at a time because of the size of the nanoparticle relative to the size of the well. Thus, clustering initiated from the single template polynucleotide in the well would result in formation of a monoclonal cluster within the well.

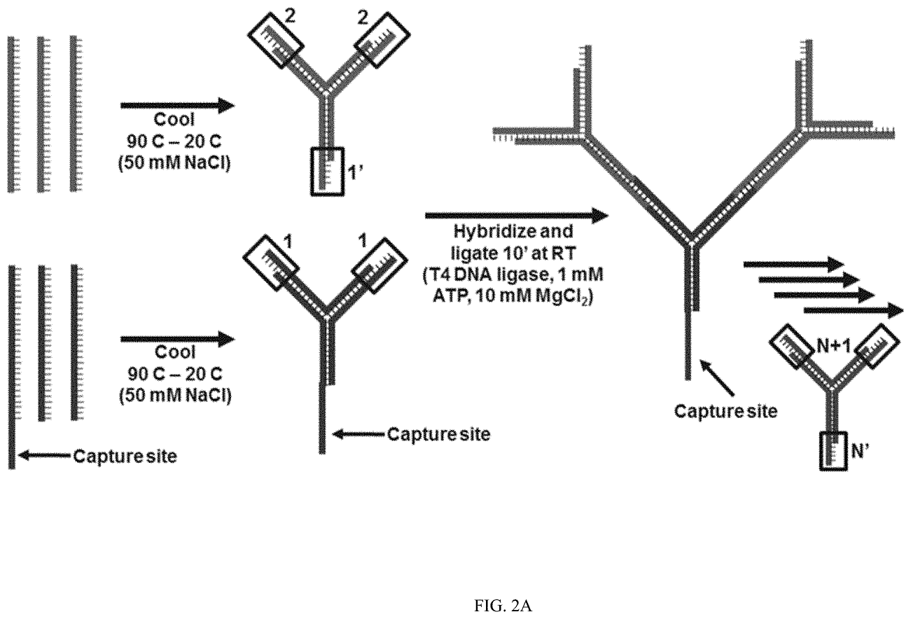

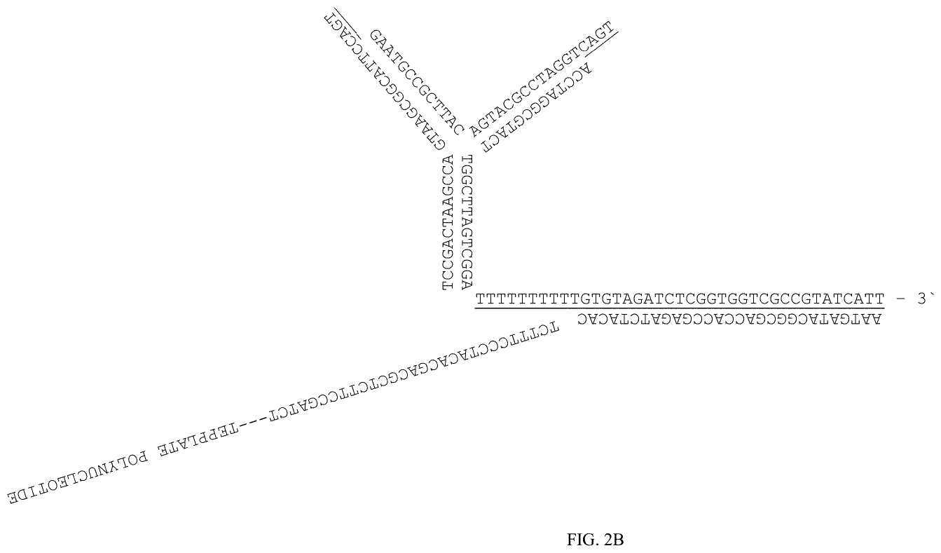

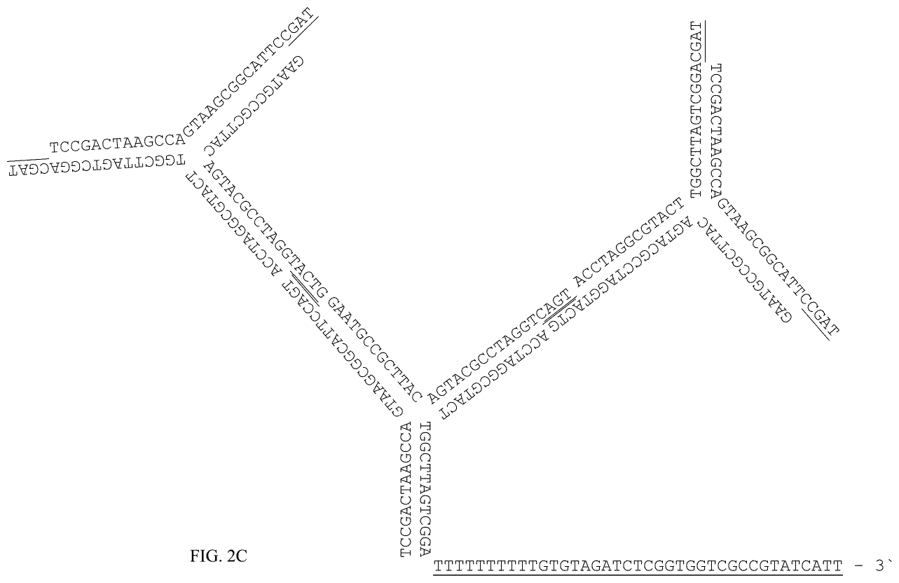

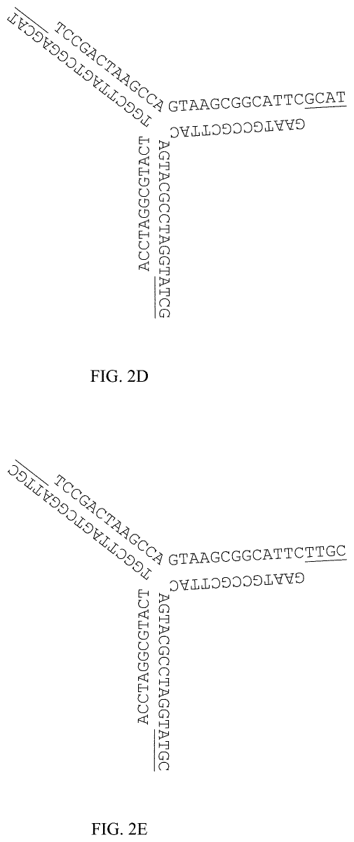

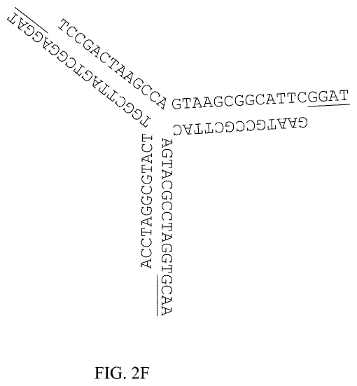

FIG. 2A shows a non-limiting working example of a DNA scaffold. In this non-limiting example, the DNA scaffold includes a plurality of scaffold DNA molecules, wherein the plurality of scaffold DNA molecules forms a DNA dendrimer. The DNA dendrimer includes a number of generations of bifurcating constitutional repeating units, also referred to as adapters. A first generation is shown upstream of a second generation. An adapter of the first generation is shown on the bottom and adapters of the second generation is shown above. Each adapter includes constitutional repeating unit oligodeoxyribonucleotides hybridized to each other to form an adapter including one upstream overhang and two downstream overhangs. For the adapter of each generation, three oligo-DNA molecules hybridize to each other as shown (here, cooled 90 C to 20 C in 50 nM NaCl) to form an adapter with an upstream overhang and two downstream overhangs.

In this example, the upstream overhang of the first generation adapter is identified as a capture site, meaning a single template site for bonding a single template polynucleotide to the scaffold. The downstream overhangs of the first generation adapter 1 have a nucleotide sequence complementary to and hybridizable with the upstream overhang of the adapter of the second generation 1′. The first and second generation adapters are then hybridized to one another, resulting in attachment of the upstream overhangs of the second generation adapters 1′ to the downstream overhangs of the first generation adapter 1 due to Watson-Crick base pairing hybridization. Sequences are then ligated together, in this example for 10 minutes at room temperature in the presence of T4 DNA ligase, 1 mM ATP, and 10 mM MgCl₂. Subsequence generations may be added to and ligated to this structure as illustrated, where downstream overhangs of adapters of an added generation N′ are complementary to the downstream overhangs of the adapters of the immediately previous generation N+1. In an example, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more generations may be formed as part of a dendrimer DNA scaffold. A size of a DNA dendrimer scaffold, such as a diameter, may be controlled in part by controlling the number of generations contained in the scaffold, with more generations corresponding to a larger nanoparticle relative to a scaffold including fewer generations.

In a non-limiting example, a first generation adapter (G¹) was synthesized from the following oligonucleotide sequences (5-prime to 3-prime):

G¹a:
(SEQ ID NO: 9)
GAATGCCGCTTACAGTACGCCTAGGTCAGT;
G¹b:
(SEQ ID NO: 10)
TCCGACTAAGCCAGTAAGCGGCATTCCAGT;
and
G¹c:
(SEQ ID NO 11)
ACCTAGGCGTACTTGGCTTAGTCGGATTTTTTTTTTGTGTAGATCTCGGTG
GTCGCCGTATCATT;

wherein the underline portions of G¹a and G¹b represent downstream overhangs and the underlined portion of G¹c represents the upstream overhang. For such an example, the upstream overhang of G¹c can include the single template nucleotide site. For example, a template polynucleotide could have extending from its 5-prime end the following sequence (5-prime to 3-prime) AATGATACGGCGACCACCGAGATCTACACTCTTTCCCTACACGACGCTCTTCCGA TCT (SEQ ID NO:12). The 5-prime end of this sequence were hybridized to the 3-prime, upstream overhang of G¹c to form the structure shown in the non-limiting working example illustrated in FIG. 2B.

A non-limiting example of a second generation adapter (G²) was synthesized from the following oligonucleotide sequences (5-prime to 3-prime): G²a: GAATGCCGCTTACAGTACGCCTAGGTACTG (SEQ ID NO:13); G²b: TCCGACTAAGCCAGTAAGCGGCATTCCGAT (SEQ ID NO:14); and G²c: ACCTAGGCGTACTTGGCTTAGTCGGACGAT (SEQ ID NO:15); wherein the underlined portions of G²b and G²c represent downstream overhangs and the underlined portion of G²a represents the upstream overhang. For such an example, the ACTG upstream overhangs of each of two G²a sequences were hybridized with a downstream CATG overhang of G¹a and G¹b to form the structure shown in the non-limiting working example illustrated in FIG. 2C.

A non-limiting example of a third generation adapter (G³) was synthesized from the following oligonucleotide sequences (5-prime to 3-prime): G³a: GAATGCCGCTTACAGTACGCCTAGGTATCG (SEQ ID NO:16); G³b: TCCGACTAAGCCAGTAAGCGGCATTCGCAT (SEQ ID NO:17); and G³c: ACCTAGGCGTACTTGGCTTAGTCGGAGCAT (SEQ ID NO:18); wherein the underlined portions of G³b and G³c represent downstream overhangs and the underlined portion of G³a represents the upstream overhang. These oligonucleotide sequences were hybridized together to form the structure shown in the non-limiting working example illustrated in FIG. 2D. Upstream ATCG overhangs of each of two G³a sequences were hybridized with a downstream CGAT overhang of G²b and G²c.

A non-limiting example of a fourth generation adapter (G⁴) was synthesized from the following oligonucleotide sequences (5-prime to 3-prime): G⁴a: GAATGCCGCTTACAGTACGCCTAGGTATGC (SEQ ID NO:19); G⁴b: TCCGACTAAGCCAGTAAGCGGCATTCTTGC (SEQ ID NO:20); and G⁴c: ACCTAGGCGTACTTGGCTTAGTCGGATTGC (SEQ ID NO:21); wherein the underlined portions of G⁴b and G⁴c represent downstream overhangs and the underlined portion of G⁴a represents the upstream overhang. These oligonucleotide sequences were hybridized together to form the structure shown in the non-limiting working example illustrated in FIG. 2E. For such an example, the upstream ATGC overhangs of each of two G⁴a sequences hybridized with a downstream GCAT overhang of G³B and G³C.

A non-limiting example of a fifth generation adapter (G⁵) was synthesized from the following oligonucleotide sequences (5-prime to 3-prime): G⁵a: GAATGCCGCTTACAGTACGCCTAGGTGCAA (SEQ ID NO:22); G⁵b: TCCGACTAAGCCAGTAAGCGGCATTCGGAT (SEQ ID NO:23); and G⁵c: ACCTAGGCGTACTTGGCTTAGTCGGAGGAT (SEQ ID NO:24); wherein the underlined portions of G⁵b and G⁵c represent downstream overhangs and the underlined portion of G⁵a represents the upstream overhang. These oligonucleotide sequences were hybridized together to form the structure shown in the non-limiting working example illustrated in FIG. 2F. For such an example, the upstream GCAA overhangs of each of two G⁵a sequences hybridized with a downstream TTGC overhang of G⁴b and G⁴c. In this non-limiting example, the 3-prime end of G⁵c was attached to a fluorophore (ALEXA FLUOR 647®, Thermo Fisher Scientific) to permit visualization of the nanoparticle by fluorescent imaging.

In another example, a polynucleotide or other spacer (e.g., with a non-limiting example nucleotide sequence of CCTCCTCCTCCTCCTCCTCCTCCT (SEQ ID NO:25)) between the fluorophore and the 3-prime end of the G⁵c oligonucleotide as shown above may be included.

For a dendron with more than five generations, an adapter for the fifth generation (and every third generation thereafter as relevant) may be synthesized using oligonucleotides G⁵a, G²b, and G¹c, with the adapters for the two following generations made with oligonucleotide having the sequences of those for generation three and four, respectively.

A dendrimer DNA scaffold may be constructed from one generation to the next through the successive assembly of adapters for a given generation, hybridization thereof to the preceding generation (for adapters of a second or higher generation), and ligating the ends of oligonucleotides together where they meet upon sticky end hybridization at a boundary between an upstream adapter of one generation and a downstream adapter of the next generation.

A non-limiting example for assembling adapters and DNA dendrimers is as follows. For creating an adapter, using the relevant sequences for each generation of adapter from the non-limiting examples above, three oligonucleotides for assembling an adapter were suspended in assembly buffer (10 mM TRIS, pH 8.0; 1 mM EDTA; 50 mM NaCl) at a concentration of 200 μM. 10 μL of each solution was then combined with 20 μL of assembly buffer. The combination was denatured at 95 degrees C. for 2 min, cooled at 65 degrees C. for 2 minutes, then annealed at 60 degrees for 6 min. Thirty-nine steps of annealing followed, at 30 sec per step, with a 0.1 degrees C. reduction in temperature (starting at 59.1 degrees C.).

To attach the second generation adapter to the first, a 150 μL solution was made including T4 DNA Ligase (NEB M0202M) with 15 μL 10× ligase buffer, and brought to a total volume of 150 μL with assembly buffer an containing 0.5 μM generation 1 adapter and 2 μM generation 2 adapter. 100 μL of this reaction was then combined with 450 μl assembly buffer, then transferred to a 50 kDa MWCO filter. The sample was then centrifuged for 1 min at 15,000×g, Filtering was repeated 10 times, adding 400 μL assembly buffer for each centrifugation step. After addition of another 400 μL assembly buffer, dendrimers were eluted from the filter by placement in a new tube upside down and centrifuging for 3 min. at 5,000×g. Volume was then brought up to 100 μl by addition of assembly buffer

Adapters for the third generation of dendrimer were then added to the generation 1-to-generation 2 dendrimer in a solution at a ratio of 4 μM to 0.5 with T4 DNA ligase and ligase buffer brought to approximately 60 μL volume with assembly buffer. Generation 4 adapters were added to the generation 1-to-generation 3 dendrimer in a solution at a ratio of 8 μM to 0.5 μM with T4 DNA ligase and ligase buffer brought to approximately 63 μL volume with assembly buffer. Generation 5 adapters were added to the generation 1-to-generation 4 dendrimer in a solution at a ratio of 15 μM to 0.5 μM with T4 DNA ligase and ligase buffer brought to approximately 69 μL volume with assembly buffer. Generation 6 adapters were added to the generation 1-to-generation 5 dendrimer in a solution at a ratio of 22.5 μM to 0.5 μM with T4 DNA ligase and ligase buffer brought to approximately 75 volume with assembly buffer. Generation 6 adapters were added to the generation 1-to-generation 5 dendrimer in a solution at a ratio of 22.5 μM to 0.25 μM with T4 DNA ligase and ligase buffer brought to approximately 75 μL volume with assembly buffer.

In an example, size of a DNA dendrimer scaffold may be determined as a function a number of generations of adapters it includes. For example, dendron DNA scaffolds having from 2 to 9 generations were synthesized as described above and their diameters measured by DLS. Results are shown in Table 2:

TABLE 2
Diameters of Dendron DNA scaffolds as
a function of number of generations

| Generations | Diameter (nm) |
|---|---|
| 9 | 198.3 |
| 8 | 167.7 |
| 7 | 158.1 |
| 6 | 142.0 |
| 5 | 89.2 |
| 4 | 99.0 |
| 3 | 59.1 |
| 2 | 31.2 |

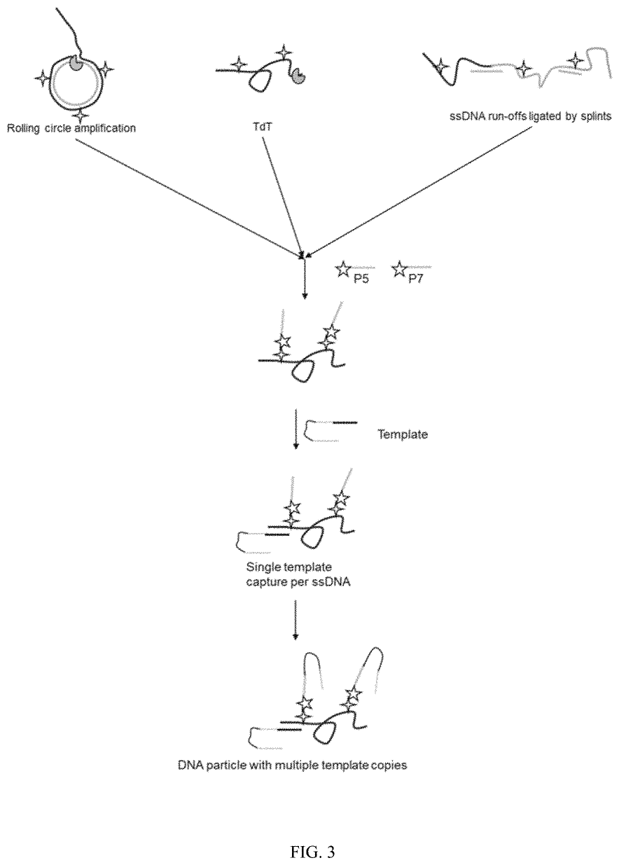

FIG. 3 illustrates nonlimiting examples of an ssDNA scaffold. In this non-limiting example, different ways of synthesizing an ssDNA scaffold are shown. A single template site is present on an end of the ssDNA scaffold. Four-pointed stars indicate accessory sites on the scaffold. P5 and P7 represent accessory oligonucleotides, and five-pointed stars represent moieties or structures complementary to the accessory sites. In an example shown at the top left, a circular DNA template is used as a template for synthesis of an ssDNA molecule by a DNA polymerase, in a rolling circle amplification process. Replication of a strand by, for example, a strand-displacing DNA polymerase (e.g., Phi29) may produce an ssDNA molecule including concatemerized copies of the copied strand of the circular coding strand. Size of the ssDNA scaffold may be determined in part by controlling the size of the circular template and a duration of a rolling circle amplification process (with a longer duration of polymerization during the rolling circle amplification process yielding a longer ssDNA scaffold).

Another non-limiting example, shown in the top middle, includes synthesis of an ssDNA template by use of a template-independent polymerase (e.g., terminal deoxynucleotidyl transferase, or TdT). Template-independent polymerases such as TdT incorporate deoxynucleotides at the 3-prime-hydroxyl terminus of a single-stranded DNA strand, without requiring or copying a template. Size of an ssDNA synthesized by use of a template-independent polymerase may be controlled by modifying a duration of a polymerization process during which a scaffold is synthesized.

Another non-limiting example of a method for synthesizing an ssDNA scaffold is shown at the top right. In this example, several single-stranded DNA molecules are synthesized by whatever method desired. In an example, an ssDNA molecule is synthesized in a run-off polymerization process, where a polymerase proceeds along a coding strand such from a linearized plasmid synthesizing a nascent strand complementary thereto until it reaches the end of the linear coding strand. Upon reaching the end the polymerase runs off the end of the coding strand any synthesis of the ssDNA molecule is completed. A plurality of ssDNA products may be synthesized, then ligated end-to-end for formation of a single ssDNA scaffold including each of the plurality. In an example, ligation of one ssDNA product to another may be accomplished with the aid of a splint, as shown. For example, a short oligo-DNA may be designed whose 3-prime end is complementary of the 5-prime end of one ssDNA product and whose 5-prime end is complementary to the 3-prime end of another ssDNA product, such that hybridization of the DNA-oligo to the two ssDNA products brings the 5-prime end of one together with the 3-prime end of the other in a nicked, double-stranded structure where they meet hybridized to the DNA-oligo. A DNA ligase (e.g., T4) may then be used to enzymatically ligate the two ends together to form a single ssDNA molecule from the two. Additional reactions may be included with DNA-oligos for splint-aided ligation of one or both ends of the product of such first reaction to another ssDNA product, and so on, for construction of an ssDNA scaffold as may be desired. Size of an ssDNA made in this way may be controlled by controlling the number and size of ssDNA molecules that are ligated together to form the ssDNA scaffold. These examples are no exhaustive. They are also not mutually exclusive, as more than one or all three may be used together in synthesis of an ssDNA scaffold.

In this non-limiting example, accessory oligonucleotides are shown bonding to the ssDNA scaffold. The accessory oligo-DNAs may bond to accessory sites by any of the various methods for doing so disclosed herein. A 5-prime end of a template is then shown bonding to the single template site at the complementary 3-prime end of the scaffold by non-covalent Watson-Crick base pairing hybridization. A clustering process is then performed on the scaffold. Ends of the portion of the template polynucleotide not hybridized to the ssDNA scaffold contain sequences corresponding to or complementary to the P5 and P7 accessory oligonucleotides. Following multiple rounds of polymerization, a scaffold-bound complement to the template polynucleotide and a scaffold-bound copy of the template polynucleotide can be seen, having been extended from the 5-prime ends of the P5 and P7 accessory oligonucleotides. In this example, a first polymerization did not displace the 5-prime end of the template polynucleotide from hybridizing to the 3-prime end of the scaffold. Thus, a sequence complementary to the portion of the 5-prime end of the template polynucleotide complementary to the hybridized end of the ssDNA scaffold was not included in the scaffold-bound complement to the template polynucleotide synthesized.

And of the above-disclosed moieties or structures for bonding a template polynucleotide, or an accessory such as an accessory oligonucleotide, to a DNA scaffold as disclosed herein may be used for bonding a template polynucleotide or an accessory to a scaffold. In some examples, commercially available nucleotides bearing such moieties or structures, including an azide group, an alkyne group, a cyclooctyne group, a biotin group, or a thiol group and capable of being incorporated into a nascent DNA strand may be included in a DNA scaffold. For example, in a polymerase reaction during which a DNA scaffold is synthesized, modified nucleotides may be seeded into the polymerization reaction at a chosen concentration relative to the concentration at which non-modified nucleotides are present. Depending on such concentration, a certain percentage of nucleotides incorporated into the DNA scaffold will be the modified nucleotides. More than one type of modified nucleotide may be seeded into a reaction, for inclusion of more than one type of moiety or structure for bonding to the DNA scaffold by compositions possessing moieties or structures complementary thereto. By incorporating modified nucleotides into a DNA scaffold, or modified nucleotides capable of being further modified for addition thereto of moieties or structures as disclosed herein for bonding between a scaffold an accessory or template polynucleotide, single template sites and accessory sites may be included in a DNA scaffold.

In an example, a nucleotide may be modified so as to include a linker such as a polyethylene glycol or other linker to another nucleotide such as a nucleotide of a polynucleotide to which it is linked. In another example, a nucleotide may be modified so as to include a linker such as a polyethylene glycol or other linker to an amino acid such as an amino acid of a polypeptide to which it is linked. Such linked-to polynucleotide or linked-to polypeptide may be a bonding site for a template polynucleotide or accessory, such as trough the examples of noncovalent bonding disclosed herein.

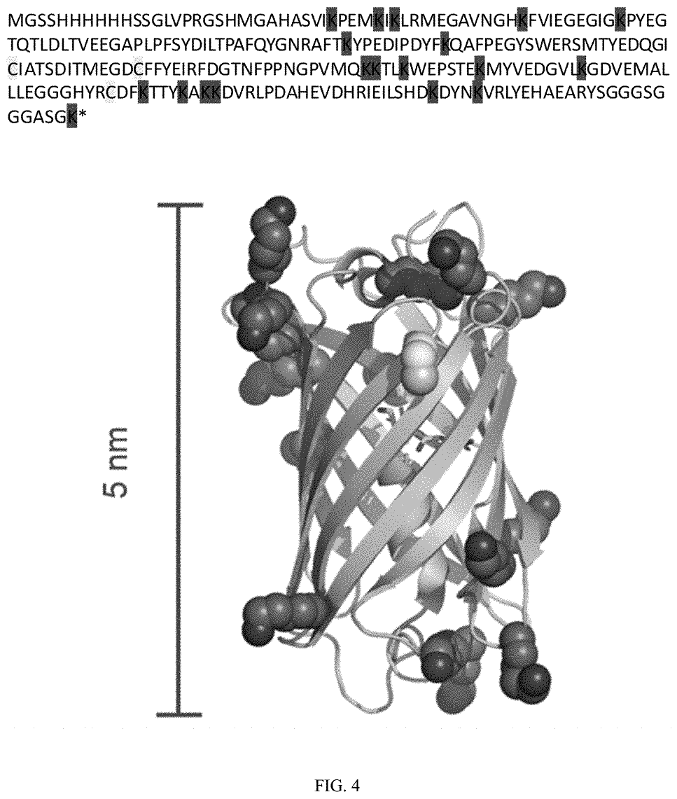

FIG. 4 shows an illustration of a non-limiting working example of a polypeptide scaffold. In this example, a green fluorescent protein (GFP) with the amino acid sequence and conformation shown in FIG. 4 was used. GFP contains three cysteine residues. When GFP adopts a three-dimensional structure as illustrated in FIG. 4, only one of the cysteines, indicated as C137 in the sequence presented in FIG. 4, is exposed as outwardly facing from the molecule as illustrated in FIG. 4. Cysteine residues at C125 and C195 may be buried within the three-dimensional conformation of GFP as and not exposed on the outside of the structure as shown and may therefore be unavailable for bond formation. When only a single cysteine residue is available for bond formation (e.g., outwardly facing C137 of the structure depicted in FIG. 4), the thiol group may serve as the single template polynucleotide site of a GFP protein scaffold.

In two other examples, C125 and C195 were mutated, both to alanine in an example and both to valine in another example, by standard recombinant methods to leave only a single thiol site as a scaffold template nucleotide bonding site, at C137. Such single cysteine residue, with its thiol group, may be a single template nucleotide site, because the GFP protein scaffold lacks other thiol groups, having only one thereof, and several possibilities of moieties or structures that can form bonds with such a thiol group as a moiety or structure complementary thereto may be used for bonding a template polynucleotide thereto. A GFP protein scaffold may also include numerous lysine residues (e.g., 19 as shown in the sequence illustrated in FIG. 4). Lysine residues include amine groups of their side chains. Amine groups of lysine residues in a GFP scaffold may therefore serve as accessory sites, and accessories such a oligo-DNA molecules, or polymers, that contain moieties or structures that can bond to such amine groups may be present on such accessories for bonding to the lysine amine group accessory sites.

In another example, amine groups of a polypeptide scaffold such as GFP may be effectively transformed into other attachment sites. In an example, bifunctional linkers having an NHS-ester at one end and an azide group at the other, separated by a PEG24 sequence, were attached to amine sites of a GFP polypeptide scaffold. The NETS-ester ends of the bifunctional linkers bonded with the amine groups of the GFP polypeptide scaffold, leaving azide groups exposed available as accessory bonding sites. The additions results in an increase in size of approximately 20 kDa of the GFP polypeptide scaffold as measured by gel electrophoresis, consistent with addition of 20 bifunctional linkers (each being 1157 Da in size), one to each of the amine groups of the 19 lysine residues and one to the N-terminal of the GFP polyprotein scaffold. In other examples, different bifunctional linkers could be used for effectively replacing a thiol site, or effectively replacing the amine groups or thiol group with different moieties or structures.

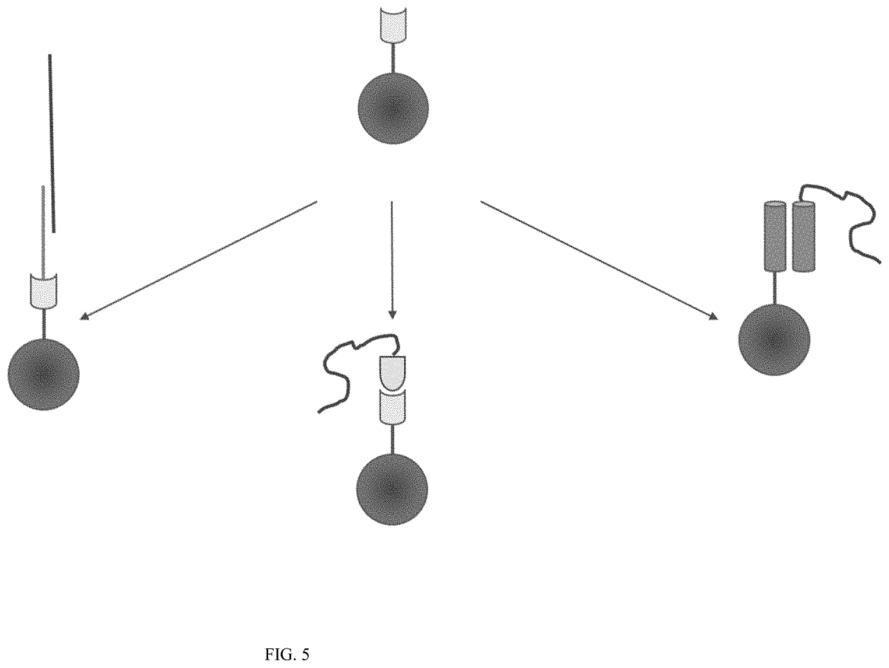

FIG. 5 is an illustration of different methods for bonding a template polynucleotide to a scaffold. At the top a scaffold is shown with a single template polynucleotide site. To the left, a template site primer is included in the single template polynucleotide site, and end of which is complementary to an end of a template polynucleotide, to permit non-covalent bonding of a template polynucleotide to the scaffold by Watson-Crick base pair hybridization. In the middle, a template polynucleotide and the single template polynucleotide site possess respectively complementary moieties or structures resulting in formation of a covalent bond forming between the template polynucleotide and the scaffold. On the right, a polypeptide is included in the single template site, and a polypeptide complementary thereto is attached to an end of the template polynucleotide. Noncovalent bonding between the polypeptide of the single template site and the template polynucleotide bonds a template polynucleotide to the scaffold.

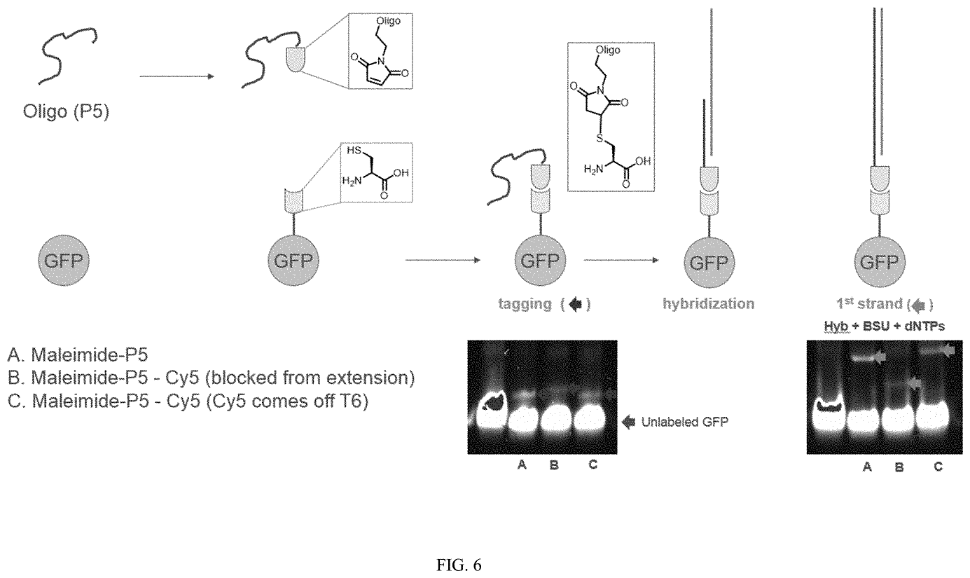

FIG. 6 illustrates a non-limiting working example in which a template polynucleotide was bonded to a protein scaffold by hybridization to a primer extending from the single template site of the scaffold, then the primer was extended by a polymerase forming a scaffold-bound complement to the template polynucleotide. A maleimide moiety was attached to the 5-prime end of a P5 oligonucleotide. The P5 oligonucleotide was attached to the single accessible template site of a GFP polypeptide scaffold, by a thiol-maleimide bond between the maleimide group of the oligo and the thiol group of the accessible cysteine residue of the GFP scaffold. The migrating band indicated by arrows (tagging) in the polyacrylamide gel electrophoresis (PAGE) blot on the left shows thiol-maleimide bonding between the scaffold and the P5 oligonucleotide. A template polynucleotide, whose 3-prime end was complementary to the P5 oligonucleotide, bonded to the scaffold by non-covalent hybridization to the P5 oligonucleotide. A polymerization reaction was then performed to form a scaffold-bound to form a scaffold-bound complement to the template polynucleotide.

The PAGE blot on the right show results from polymerization reactions run under three different conditions. In condition A, the P5 oligonucleotide was bound to the scaffold by a thiol-maleimide bond. The band indicated by an arrow (1st strand) in column A indicates that a scaffold-bound complement to the template polynucleotide was formed on the scaffold during a polymerization. In column B, extension of the P5 oligonucleotide was prevented by attachment of a Cy5 fluorophore in a blocking position on the 3-prime nucleotide of the P5 oligonucleotide preventing it from being extended by a polymerase. The arrow in column B indicates that a scaffold-based complement to the template polynucleotide was not formed, confirming the positive result shown in column A. In column C, Cy5 was bound to the P5 oligonucleotide via a hexathymidine (T6) without an extension block. The arrow in column C, matching the arrow in column A, confirms again that a scaffold-bound complement to the template polynucleotide was formed and that the absence thereof in column B was not the result of a false negative due merely to the presence of Cy5.

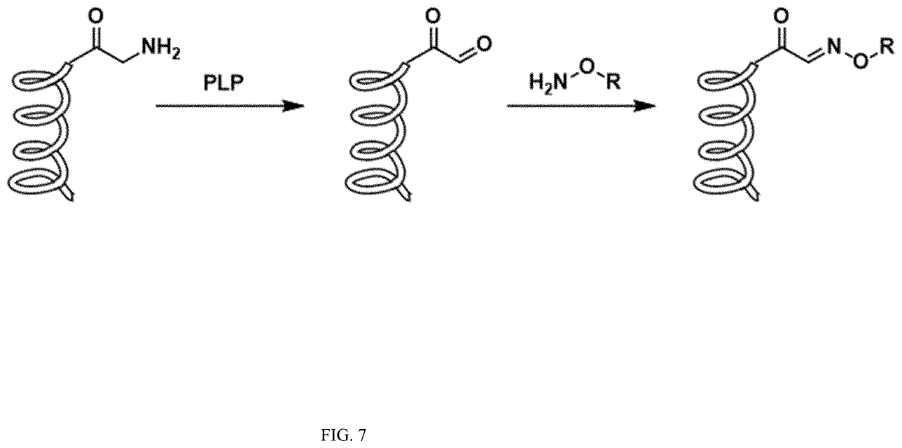

FIG. 7 shown a non-limiting example of a bioconjugation including (PLP)-mediated transamination specific for the N-terminus of a protein. The reaction oxidizes the N-terminal amine to a ketone or an aldehyde, which then forms a stable oxime linkage with an alkoxyamine.

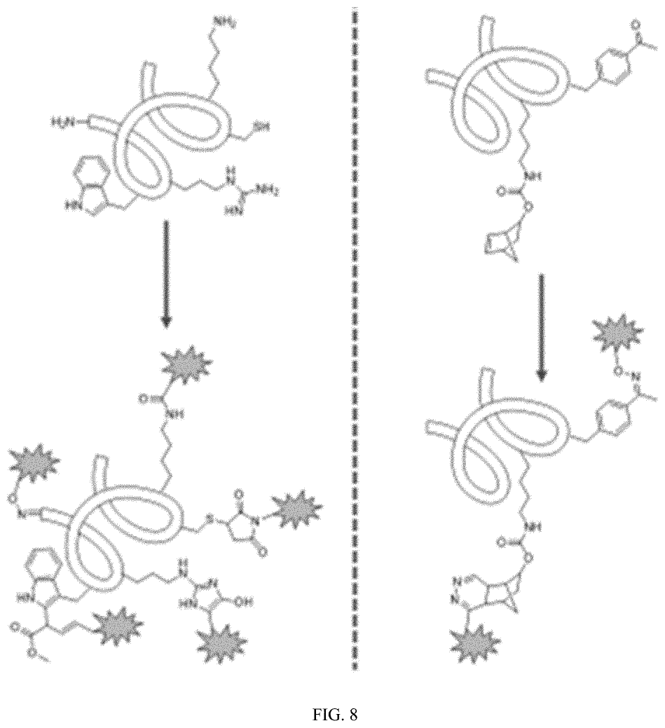

FIG. 8 shows non-limiting examples of bonds that can form with natural amino acids to bond to accessory sites of a scaffold, as shown here, or also for a single template site. Examples include thiol-maleimide coupling to a cysteine residue, amine-NETS bonding at a lysine residue, rhodium carbenoids bonding to a tryptophan residue, alpha,beta-dicarbonyl bonding to an arginine residue, and PLP-mediated transamination of a protein N-terminus followed by oxime bond formation. On the right is shown examples of site-specific modification of proteins at modified, non-natural occurring amino acid residues. One example shows a non-natural ketone amino acid reacting with a hydroxyamine to form an oxime. The second reaction shows and non-natural norbornene (or other strained alkene/alkyne) reacting with a tetrazine.

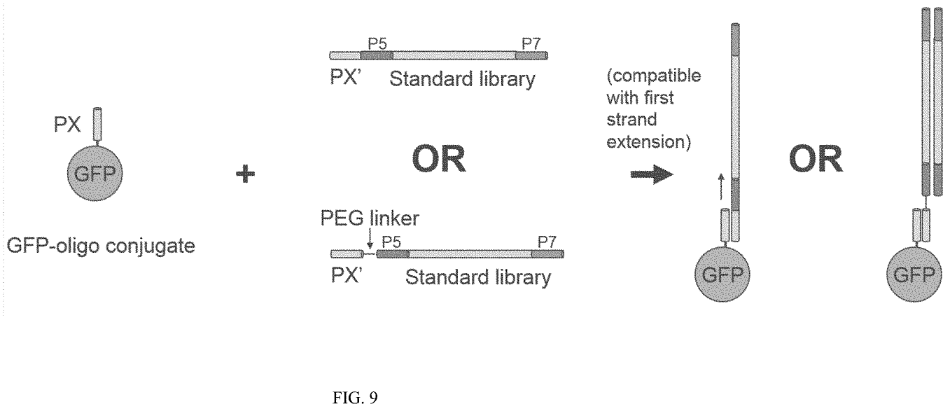

FIG. 9 shows examples of a template polynucleotide bonded to a single template site of a protein scaffold by hybridization to a template site primer represented by PX (GFP-oligo conjugate). Two examples of template polynucleotides of a library are shown, one a standard library molecule with P5 and P7 sequences, with a region complementary to the PX template site primer (denominated PX′) at the 3-prime end, or a modified version where the PX′ sequence is separated from the P5 sequence by a PEG linker. Ether strand bonds to a single template site of a scaffold. The standard library sequence can be used in a first strand extension polymerization reaction, with the PX primer serving as an initiation primer for polymerization of a nascent strand complementary to the template polynucleotide.

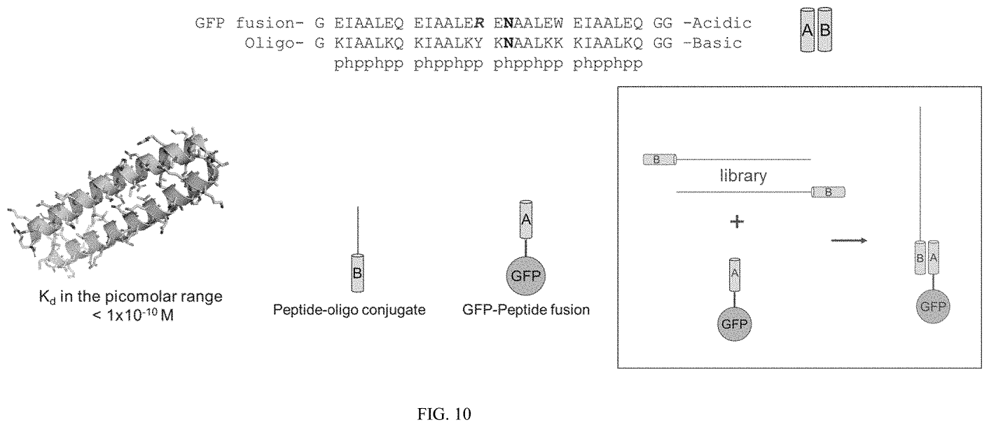

FIG. 10 shows an example of a non-covalent bond, specifically a coiled coil peptide non-covalent bond (in an example, with K_{D} in the picomolar range <1×10⁻¹⁰ M). Two amino acid sequences for alpha helical polypeptide structures are shown that form two complementary bonding partners of a coiled coil attachment. By attaching one such sequence to the scaffold such as to a polypeptide scaffold as illustrated in FIG. 10 (GFP-Peptide fusion) and the other to template polynucleotide (peptide-oligo conjugate) or to a library template polynucleotide, the template polynucleotide can be bound to the scaffold via non-covalent bonding between the alpha helices. In another example, one of the alpha helical sequences complementary to that attached to the scaffold can be attached to an accessory such as an accessory oligonucleotide for attachment of an accessory oligonucleotide to an accessory site.

FIG. 11 shows selective bioconjugation of lysine side chains of a polypeptide scaffold using activated NETS-esters. Lysine was bound to dibenzocyclooctynes (DBCO). The alkyne moiety of DBCO can be subsequently appended with azide-containing molecules via a strain-promoted [3+2] cycloaddition click reaction. Molecules that can subsequently be appended to the DBCO motif include, as non-limiting examples, oligonucleotides and polymers. A GFP polypeptide scaffold was readily labeled with DBCO producing a mix of DBCO labeled scaffolds. No unlabeled GFP was detected by SDS PAGE gel analysis of the products of the reaction (not shown).

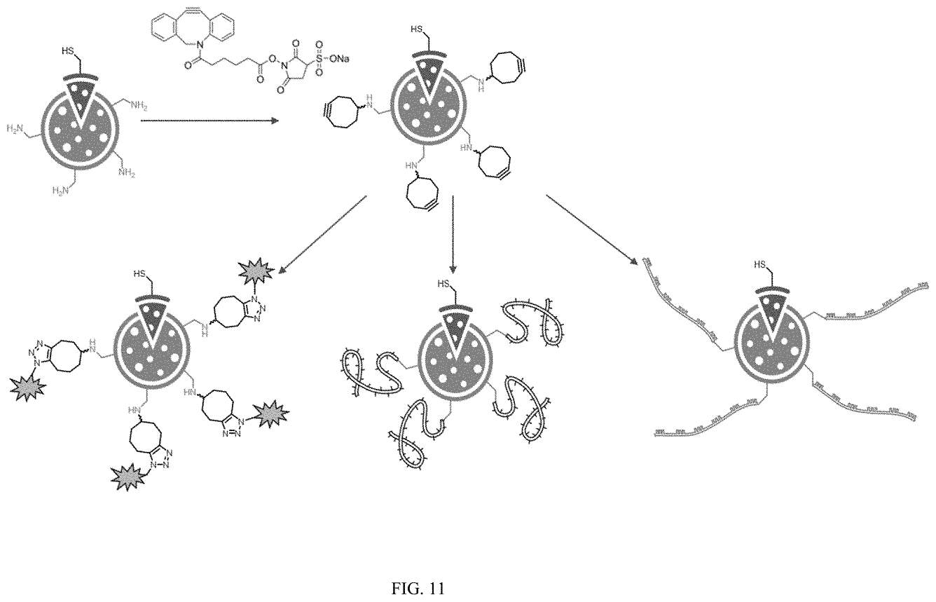

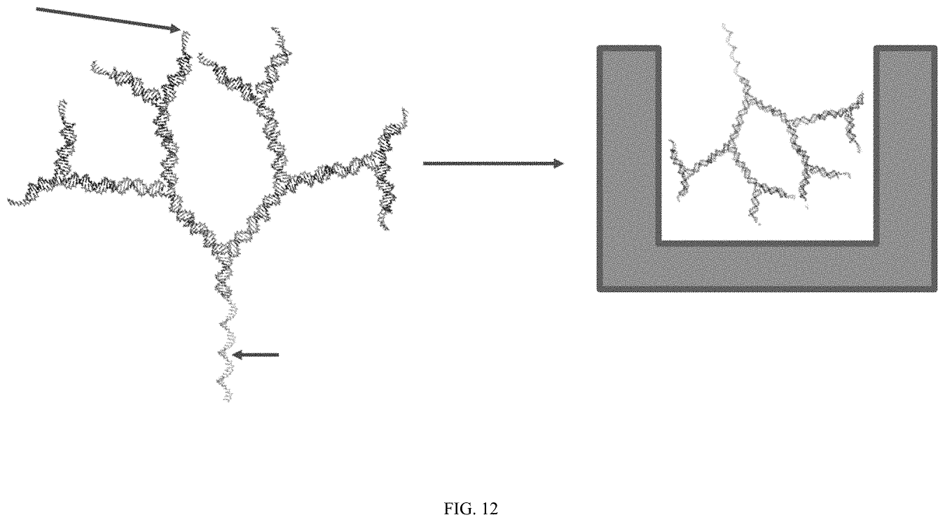

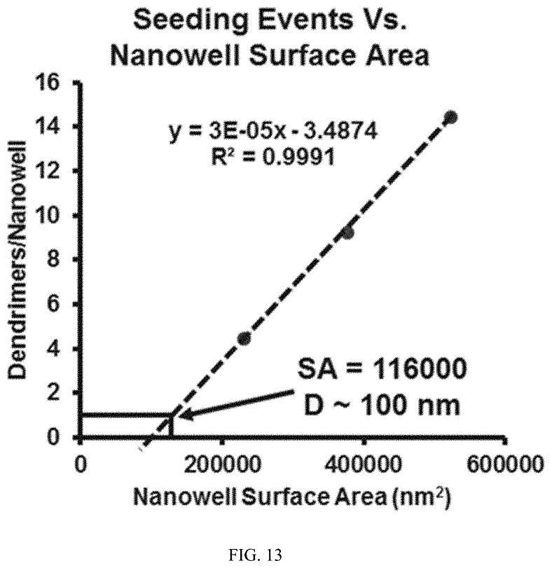

FIG. 12 shows an example illustration of a non-limiting working example of a scaffold with a single template polynucleotide attached to it attaching to a surface of a well of a flow cell. In this non-limiting example, the scaffold is a DNA dendrimer. The single template site extends from the upstream overhang of the first generation adapter and the accessory sites the downstream overhangs of the last generation of adapters. FIG. 13 shows a graph (Seeding Events vs. Nanowell Surface Area) of a non-limiting working example of tests of numbers of scaffolds of a given size that can be present in a nanowell (Dendrimers/Nanowell) of a given nanowell surface area (SA) or diameter (D). DNA dendrimer nanoparticles of about 100 nm in diameter were seeded into nanowells 185, 285, or 375 nm in diameter and the number of dendrimers per nanowell measured. Extrapolating from the best fit curve of the results (y=3E-0.5x−3.4874, R²=0.9991) indicates that single-nanoparticle seeding of a nanowell would result, in this examples, of using a nanoparticle with a diameter of about 100 nm and a nanowell with a diameter of about 100 nm.

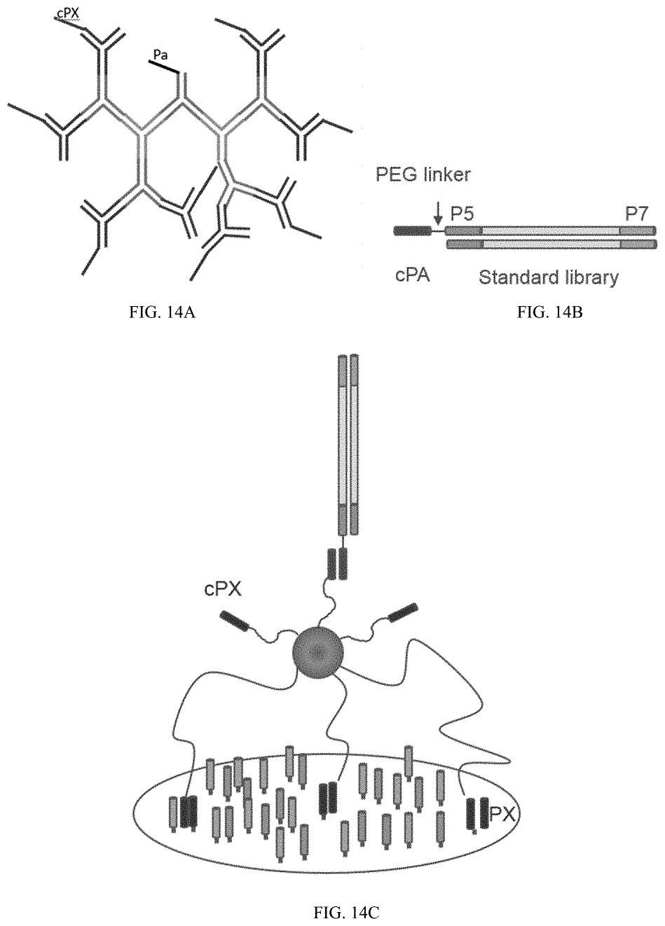

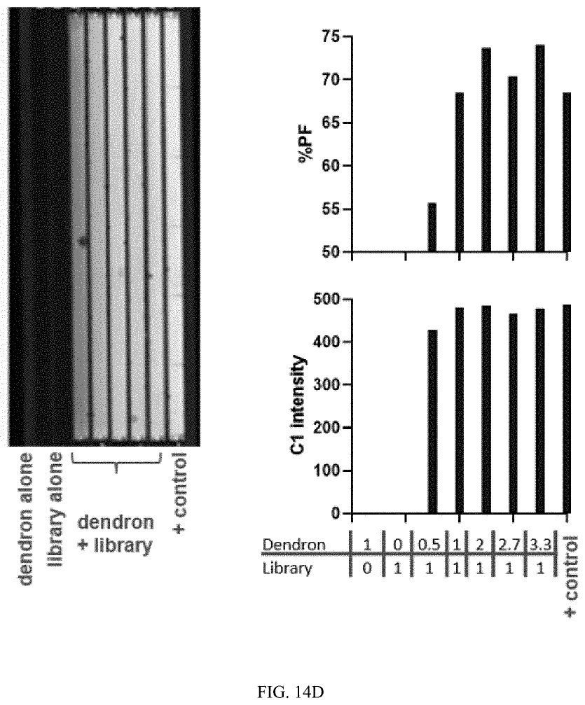

FIGS. 14A-14D show an example of seeding a substrate with template polynucleotides using a DNA scaffold in accordance with aspects of the present disclosure. FIG. 14A shows a depiction of a scaffold DNA molecule including a DNA dendrimer in accordance with the present disclosure. Scaffolds had a diameter of from 50 nm-150 nm. The scaffold includes a single template site (Pa) for bonding a template polynucleotide and a plurality of accessory sites (cPX). FIG. 14B shows a template polynucleotide and its complement, with a primer sequence added to each end (P5/cP5 and P7/cP7). The P5-primer end of the template polynucleotide is connected to a primer (cPa) by a PEG linker. The cPa primer is complementary to the single template site (Pa) of the scaffold DNA molecule depicted in FIG. 14A. FIG. 14C is a depiction of the scaffold DNA molecule depicted in FIG. 14A hybridized, via its single template site (Pa), to the template polynucleotide and its complement depicted in FIG. 4B, via the cPa primer. The scaffold is attached to a substrate. The substrate is attached to primers (PX) that are complementary to accessory sites (cPX).of the scaffold. The substrate is also attached to primers that to permit hybridization of template ends thereto to permit clustering on the substrate

FIG. 14D depicts an example according to the foregoing demonstrating seeding a substrate with a template polynucleotide using a scaffold with a single template site followed by clustering. Scaffolds attached to template polynucleotides according to the present disclosure and FIGS. 14A-14C. Dendrimer scaffolds were combined with template polynucleotides (library) at the molar rations shown, a substrate (flow cell with nanowells for seeding) seeded therewith, then clustering performed according to a recombinase-driven cluster amplification process (ExAmp cluster amplification). Negative controls include scaffold without template and template without scaffold. As a positive control (+ control), clustering on substrate was performed without dendron, using clustering on substrate following hybridization of template molecules to primers attached to the substrate not via a scaffold.

The left panel is an image of a flow cell following a clustering process according to the above conditions (2 negative controls, 5 conditions of various scaffold:template molar ratios, and 1 positive control). Fluorescence in all conditions except the negative controls indicate that a scaffold with a single template binding site can seed a substrate with a template polynucleotide and support a clustering process. Bar graphs are quantitative measurements of clustering results of the 8 conditions. Upper graph, C1 intensity is cycle 1 intensity as an indirect measure of the cluster size or yield (with intensity being directly proportional to cluster size or yield). Lower graph, % PF is % passing filter, which is the percent of nanowells passing a threshold filter indicating purity of cluster formed therein, i.e. directly proportional to number of nanowells with monoclonal clusters.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

What is claimed is:

1. A nanoparticle, comprising
a scaffold, a single template site for bonding a template polynucleotide to the scaffold, and a plurality of accessory sites for bonding accessory oligonucleotides to the scaffold, wherein
the scaffold is selected from one or more scaffold DNA molecules and one or more scaffold polypeptides,
the single template site for bonding a template polynucleotide to the scaffold is selected from a covalent template bonding site and a noncovalent template bonding site and
the plurality of accessory sites for bonding accessory oligonucleotides to the scaffold is selected from covalent accessory oligonucleotide bonding sites and noncovalent accessory oligonucleotide bonding sites.

2. The nanoparticle of claim 1, wherein the scaffold comprises one or a plurality of scaffold DNA molecules.

3. The nanoparticle of claim 2, wherein the scaffold comprises a plurality of scaffold DNA molecules, wherein the plurality of scaffold DNA molecules comprises a DNA dendrimer.

4. The nanoparticle of claim 3, wherein the DNA dendrimer comprises a number of generations of bifurcating constitutional repeating units wherein the number of generations is from 2 to 100.

5. The nanoparticle of claim 4, wherein the bifurcating constitutional repeating units each comprise three constitutional repeating unit oligodeoxyribonucleotides hybridized to each other to form an adapter comprising one upstream overhang and two downstream overhangs, wherein the upstream overhang of each adapter in generation 2 and higher is complementary to a downstream overhang of an immediately upstream constitutional repeating unit, and the downstream overhang of the adapter in generation 1 comprises the single template site.

6. The nanoparticle of claim 2, wherein the scaffold comprises a single-stranded DNA.

7. The nanoparticle of claim 1, wherein the scaffold comprises one or more scaffold polypeptides.

8. The nanoparticle of claim 7, wherein the scaffold polypeptide comprises a green fluorescent protein.

9. The nanoparticle of claim 1, wherein the single template site comprises a covalent template bonding site.

10. The nanoparticle of claim 1, wherein the single template site comprises a noncovalent template bonding site.

11. The nanoparticle of claim 10, wherein the noncovalent template bonding site comprises a polynucleotide hybridization site.

12. The nanoparticle of claim 10, wherein the noncovalent template bonding site is selected from a noncovalent peptide binding site and the noncovalent peptide binding site is selected from a coiled-coil bonding site and an avidin-biotin bonding site.

13. The nanoparticle of claim 1, wherein the plurality of accessory sites for bonding accessory oligonucleotides to the scaffold comprise covalent accessory oligonucleotide bonding sites.

14. The nanoparticle of claim 1, wherein the accessory oligonucleotide bonding sites comprise noncovalent accessory oligonucleotide bonding sites.

15. The nanoparticle of claim 14, wherein the noncovalent accessory oligonucleotide bonding sites comprise polynucleotide hybridization sites.

16. The nanoparticle of claim 14, wherein the noncovalent accessory oligonucleotide bonding sites comprise noncovalent peptide binding sites and the noncovalent peptide binding sites are selected from one or both of coiled-coil bonding sites and avidin-biotin bonding sites.

17. A method, comprising bonding a single template polynucleotide to the single template site of the nanoparticle of claim 1.

18. A method, comprising bonding a plurality of accessory oligonucleotides to the plurality of accessory sites of the nanoparticle of claim 1.

19. The method of claim 17, further comprising attaching the scaffold to a substrate, wherein attaching comprises hybridizing accessory oligonucleotides with oligonucleotides attached to the substrate.

20. The method of claim 25, wherein the substrate comprises a plurality of nanowells and the oligonucleotides attached to the substrate are attached within the plurality of nanowells.
